(19) United States
(12) Patent Application Publication
(10) Pub. No.: US 2022/0162640 A1
(43) Pub. Date: May 26, 2022

(54) HYBRID PROMOTERS FOR MUSCLE EXPRESSION

(71) Applicants: GENETHON, EVRY (FR); INSTITUT NATIONAL DE LA SANTE ET DE LA RECHERCHE MEDICALE (INSERM), PARIS (FR); SORBONNE UNIVERSITE, PARIS (FR); UNIVERSITE D'EVRY VAL D'ESSONNE, EVRY (FR)

(72) Inventors: GIUSEPPE RONZITTI, FONTAINEBLEAU (FR); PATRICE VIDAL, RIS-ORANGIS (FR); FEDERICO MINGOZZI, PHILADELPHIA, PA (US)

(21) Appl. No.: 17/601,934

(22) PCT Filed: Apr. 7, 2020
(86) PCT No.: PCT/EP2020/059919

§ 371 Date: Oct. 7, 2021

(30) Foreign Application Priority Data
Apr. 8, 2019 (EP) 19305455.8

(51) Int. Cl. C12N 15/86; A61K 48/00

(52) U.S. Cl. CPC C12N 15/86; A61K 48/0058; C12N 2840/007; C12N 2750/14143; C12N 2830/008

(57) ABSTRACT

The present invention relates to hybrid promoters to drive gene expression in muscles.

### FIELD OF THE INVENTION

The present invention relates to hybrid promoters to drive gene expression in muscles. The invention further relates to expression cassettes and vectors containing said hybrid promoters. Also disclosed herein are methods implementing these hybrid promoters, in particular methods of gene therapy.

### BACKGROUND OF THE INVENTION

Neuromuscular disorders represent one of the main challenges for in vivo based gene therapy. Yet, insufficient transgene expression in the desired target tissues and anti-transgene immunity still represent important hurdles to achieve successful gene therapy for many diseases.

Therefore, there is still a need of providing strong expression of a transgene into the cell of interest, but at low dose of vectors to prevent both potential toxicity of the vector and immune response against the vector.

Theoretically, this aim could be addressed by selecting a promoter providing strong expression in the target cell. However, several problems may arise from their use. In particular, when designing a construct for gene therapy, one has to keep in mind size constraints specific to the vector used to deliver the therapeutic transgene. For example, the elements introduced into an AAV vector should have a reduced size due to the limitations posed by the maximum encapsidation size of AAV vectors, i.e. approximately 5 kb.

Here, we describe the identification of an enhancer/promoter combination with a size compatible with gene therapy vectors such as AAV vectors, allowing the efficient expression of proteins muscles.

### SUMMARY OF THE INVENTION

The present invention provides genetic engineering strategies implementing novel hybrid promoters having muscle specificity. These hybrid promoters may be used in gene therapy of neuromuscular diseases. These novel hybrid promoters are based on the combination of one or more liver-selective enhancer(s) operably linked to a muscle-selective promoter. Surprisingly, it is herein shown that the expression of a transgene is increased in muscle cells when placed under the control of such a hybrid promoter including a liver-selective enhancer.

Accordingly, the first aspect of the invention relates to a nucleic acid molecule comprising one or a plurality of liver-selective enhancer(s) operably linked to a muscle-selective promoter.

In a further particular embodiment, the nucleic acid molecule comprises one liver-selective enhancer operably linked to a muscle-selective promoter. In another embodiment, the nucleic acid molecule comprises a plurality of liver-selective enhancers operably linked to a muscle-selective promoter. In a particular embodiment, the plurality of liver-selective enhancers comprises at least two liver-selective enhancers. In yet another embodiment, the plurality of liver-selective enhancers comprises two liver-selective enhancers. In a further embodiment, the plurality of liver-selective enhancers comprises three liver-selective enhancers. In a further embodiment, the plurality of liver-selective enhancers comprises four liver-selective enhancers. In yet another embodiment, the plurality of liver-selective enhancers comprises five liver-selective enhancers. In a specific embodiment, the nucleic acid molecule comprises one, two or three liver-selective enhancers, more particularly one or three liver-selective enhancers. In a particular embodiment, all the liver-selective enhancers of the plurality of liver-selective enhancers have the same sequence or at least two of the liver-selective enhancers of the plurality of liver-selective enhancers have a different sequence. In a specific embodiment, all the liver-selective enhancers of the plurality of liver-selective enhancers have the same sequence.

In a particular embodiment of the invention of the nucleic acid of the invention, the enhancer may be a short-sized liver-selective enhancer or the plurality of liver-selective enhancers may be a plurality of short-sized liver-selective enhancers. In particular, a liver-selective enhancer for use in the invention may consist of 10 to 175 nucleotides, such as 40 to 100 nucleotides, in particular 50 to 80 nucleotides. In a particular embodiment, a liver-selective enhancer for use in the invention may consist of 70 to 75 nucleotides. In a particular embodiment, the liver-selective enhancer is the 72 nucleotide HS-CRM8 enhancer consisting of SEQ ID NO:1, or a functional fragment of SEQ ID NO:1 having a liver-selective enhancer activity. In another embodiment, the liver-selective enhancer is a functional variant of the 72 nucleotide HS-CRM8 enhancer that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional variant has a liver-selective enhancer activity. In a further embodiment, the liver-selective enhancer is a functional fragment of a sequence that consists of a sequence that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional fragment has a liver-selective enhancer activity.

Preferably, the promoter is a short-sized muscle-selective promoter. In a particular embodiment, the promoter is a CK6 promoter, CK8 promoter, Actal promoter or a synthetic promoter C5.12 (spC5.12, alternatively referred to herein as “C5.12”). In a particular embodiment, the muscle-selective promoter is a spC5.12 promoter. spC5-12 promoter. In a further particular embodiment, the spC5-12 promoter is selected from:
a sequence that consists of the sequence shown in SEQ ID NO:2, 3 or 4, in particular the sequence shown in SEQ ID NO:2, or a functional fragment of SEQ ID NO:2, 3 or 4, in particular of SEQ ID NO:2, wherein said fragment has a muscle-selective promoter activity;
a sequence that consists of a sequence that is at least 80% identical to any one of SEQ ID NO:2, 3 and 4, such as at least 85% identical, in particular at least 90% identical, more particularly at least 91%, 92%, 93%, 94%, 95%, 96%, 97%, 98% or even at least 99% identical to any one of SEQ ID NO:2, 3 and 4, in particular to SEQ ID NO:2; and
a functional fragment of a sequence that consists of a sequence that is at least 80% identical to any one of SEQ ID NO:2, 3 and 4, such as at least 85% identical, in particular at least 90% identical, more particularly at least 91%, 92%, 93%, 94%, 95%, 96%, 97%, 98% or even at least 99% identical to any one of SEQ ID NO:2, 3 and 4, in particular to SEQ ID NO:2, wherein said functional fragment has a muscle-selective promoter activity.

Optionally, the nucleic acid molecule described therein may further comprise a further enhancer, such as a muscle-selective enhancer, for example the SA195 enhancer of SEQ ID NO:34, or the MCK enhancer of SEQ ID NO:5 or a functional variant thereof having a sequence at least 80% identical to SEQ ID NO:34 or SEQ ID NO:5, such as at least 85%, 90%, 91%, 92%, 93%, 94%, 95%, 96%, 97%, 98% or even at least 99% identical to SEQ ID NO:34 or SEQ ID NO:5. In a particular embodiment, the further enhancer is located between the liver-selective enhancer or the plurality of liver-selective enhancers and the muscle-selective promoter. In a particular embodiment, no further enhancer is provided between the liver-selective enhancer or the plurality of liver-selective enhancers and the muscle-selective promoter.

The hybrid promoter of the invention may be operably linked to a transgene of interest. Accordingly, the invention further relates to an expression cassette comprising the nucleic acid molecule described herein, operably linked to a transgene of interest.

The invention further relates to a vector comprising the expression cassette described above. In a particular embodiment, the vector is a plasmid vector. In another embodiment, the vector is a viral vector. Representative viral vectors include, without limitation, adenovirus vectors, retrovirus vectors, lentivirus vectors and parvovirus vectors, such as AAV vectors. In a particular embodiment, the viral vector is an AAV vector, such as an AAV vector comprising an AAV8 or AAV9 capsid.

The invention also relates to an isolated recombinant cell comprising the nucleic acid construct according to the invention.

The invention further relates to a pharmaceutical composition comprising, in a pharmaceutically acceptable carrier, the vector or the isolated cell of the invention.

Furthermore, the invention also relates to the expression cassette, the vector or the cell disclosed herein, for use as a medicament. In this aspect, the transgene of interest comprised in the expression cassette, the vector or the cell is a therapeutic transgene.

The invention further relates to the expression cassette, the vector or the cell disclosed herein, for use in gene therapy.

In another aspect, the invention relates to the expression cassette, the vector or the cell disclosed herein, for use in the treatment a neuromuscular disorder. In particular, the neuromuscular disorder may be selected in the group consisting of muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, motor neuron diseases (e.g. amyotrophic lateral sclerosis (ALS), spinal muscular atrophy (Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4)), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphoglycerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenita paramyotonia congenita central core disease nemaline myopathy myotubular myopathy periodic paralysis).

In a further particular embodiment, the disease is Cori disease and the transgene of interest is GDE, such as a truncated form of GDE.

### LEGEND OF THE FIGURES

FIG. 1. Schematic representation of promoter/enhancer associations. The size of each element is indicated.

FIG. 2. mSeAP protein expression in muscles. C57BL/6 mice were injected with 2×10¹¹ vg/mouse of AAV9 vectors expressing the murine secreted alkaline phosphatase (mSeAP) reporter gene under the transcriptional control of spC5-12 promoter (spC5-12) or fusions of the same promoter with MCK (MCK-spC5-12), H1 and MCK (H1-MCK-spC5-12) or H3 and MCK (H3-MCK-spC5-12) enhancers. PBS-injected mice were used as controls (PBS). One month post-injection, mSeAP activity was measured in different muscles and reported as fold variation compared to the levels measured in PBS group. Statistical analyses were performed by ANOVA (*=p<0.05, n=5 per group).

FIG. 3. mSEAP expression in non-muscle tissues. Livers, brains and kidneys from C57BL/6 mice treated as described in FIG. 2 were analyzed for mSEAP activity. The mSeAP activity measured is reported as fold variation compared to the levels measured in PBS injected mice. Statistical analyses were performed by ANOVA (*=p<0.05, n=5 per group).

FIG. 4. The presence of the MCK enhancer is not required to increase transgene expression in muscle. C57BL/6 mice were injected with 4×10¹¹ vg/mouse of AAV9 vectors expressing the murine secreted alkaline phosphatase (mSeAP) reporter gene under the transcriptional control of spC5-12 promoter (spC5-12) or fusions of the same promoter with H3 (H3-spC5-12) or H3 and MCK (H3-MCK-spC5-12) enhancers. PBS-injected mice were used as controls (PBS). One month post-injection, mSeAP activity was measured in heart, diaphragm, quadriceps and triceps and reported as fold variation compared to the levels measured in PBS group. Statistical analyses were performed by ANOVA (*=p<0.05 as indicated, n=4 per group).

FIG. 5. H3 enhancer increases muscle expression when fused with different muscle-specific promoters. C57BL/6 mice were injected with 5×10¹¹ vg/mouse of AAV9 vectors expressing the murine secreted alkaline phosphatase (mSeAP) reporter gene under the transcriptional control of CK6 or CK8 promoters or enhancer-promoter combination constituted by the H3 enhancer fused with CK6 (H3-CK6) or CK8 (H3-CK8). PBS-injected mice were used as controls (PBS). Fifteen days after vectors injection, mSeAP activity was measured in heart, quadriceps and triceps and reported as fold variation compared to the levels measured in mice injected with mSeAP under the control of CK6 promoter. Statistical analyses were performed by ANOVA (*=p<0.05 as indicated, n=4 per group).

FIG. 6. H3 enhancer increases muscle expression when fused with ACTA1 muscle-specific promoters. C57BL/6 mice were injected with 4×10¹¹ vg/mouse of AAV9 vectors expressing the murine secreted alkaline phosphatase (mSeAP) reporter gene under the transcriptional control of enhancer-promoter combination constituted by the H3 enhancer fused with spC5-12 (H3-spC5-12) or Actal (H3-Actal) promoters. PBS-injected mice were used as controls (PBS). One month post-injection, mSeAP activity was measured in heart, diaphragm, quadriceps and triceps and reported as fold variation compared to the levels measured in PBS group. Statistical analyses were performed by ANOVA (*=p<0.05 vs. PBS, n=4 per group).

FIG. 7. F enhancer increases muscle expression when fused with a muscle-specific promoter. C57BL/6 mice were injected with 4×10¹¹ vg/mouse of AAV9 vectors expressing the murine secreted alkaline phosphatase (mSeAP) reporter gene under the transcriptional control of spC5-12 promoter or the combination of F enhancer and spC5-12 promoter (F-spC5-12). PBS-injected mice were used as controls (PBS). Fifteen days after vectors injection, mSeAP activity was measured in quadriceps and reported as fold variation compared to the levels measured in PBS group. Statistical analyses were performed by ANOVA (*=p<0.05 as vs. spC5-12, n=3-4 per group).

### DETAILED DESCRIPTION

### Definitions

In the context of the present invention, a “transcription regulatory element” is a DNA sequence able to drive or enhance transgene expression in a tissue or cell.

In the context of the present invention, the expression “muscle-selective promoter” includes natural or synthetic muscle-selective promoters. In addition, the expression “liver-selective enhancer” includes natural or synthetic liver-selective enhancers.

According to the present invention tissue-selectivity means that a transcription regulatory element preferentially drives (in case of a promoter) or enhances (in case of an enhancer) expression of a gene operably linked to said transcription regulatory element in a given tissue, or set of tissues, as compared to expression in another tissue(s). This definition of “tissue-selectivity” does not exclude the possibility for a tissue-selective transcription regulatory element (such as a muscle-selective promoter) to leak to some extent. By “leak”, “leaking” or declinations thereof, it is meant the possibility for a muscle-selective promoter to drive or increase expression of a transgene operably linked to said promoter into another tissue, although at lower expression levels. For example, a muscle-selective promoter may leak in the liver tissue, meaning that expression drove from this promoter is higher in the muscle tissue than in the liver tissue. Alternatively, the tissue-selective transcription regulatory element may be a “tissue-specific” transcription regulatory element, meaning that this transcription regulatory element not only drives or enhances expression in a given tissue, or set of tissues, in a preferential manner, but also that this regulatory element does not, or does only marginally, drive or enhance expression in other tissues.

According to the present invention, a “transgene of interest” refers to a polynucleotide sequence that encodes a RNA or protein product and that may be introduced into a cell for a sought purpose, and is capable of being expressed under appropriate conditions. A transgene of interest may encode a product of interest, for example a therapeutic or diagnostic product of interest. A “therapeutic transgene” is selected and used to lead to a desired therapeutic outcome, in particular for achieving expression of said therapeutic transgene into a cell, tissue or organ into which expression of said therapeutic transgene is needed. Therapy may be achieved by a number of ways, including by expressing a protein into a cell that does not express said protein, by expressing a protein into a cell that expresses a mutated version of the protein, by expressing a protein that is toxic to the target cell into which it is expressed (strategy used, for example, for killing unwanted cells such as cancer cells), by expressing an antisense RNA to induce gene repression or exon skipping, or by expressing a silencing RNA such as a shRNA whose purpose is to suppress the expression of a protein. The transgene of interest may also encode a nuclease for targeted genome engineering, such as a CRISPR associated protein 9 (Cas9) endonuclease, a meganuclease or a transcription activator-like effector nuclease (TALEN). The transgene of interest may also be a guide RNA or a set of guide RNAs for use with the CRISPR/Cas9 system, or a correcting matrix for use in a targeted genome engineering strategy along with a nuclease as described beforehand. Other transgenes of interest include, without limitation, synthetic long non-coding RNAs (SINEUPs; Carrieri et al., 2012, Nature 491: 454-7; Zucchelli et al., 2015, RNA Biol 12(8): 771-9; Indrieri et al., 2016, Sci Rep 6: 27315) and artificial microRNAs. Other specific transgene of interest useful in the practice of the present invention are described below.

According to the present invention, the term “treatment” includes curative, alleviation or prophylactic effects. Accordingly, a therapeutic and prophylactic treatment includes amelioration of the symptoms of a disorder or preventing or otherwise reducing the risk of developing a particular disorder. A treatment may be administered to delay, slow or reverse the progression of a disease and/or of one or more of its symptoms. The term “prophylactic” may be considered as reducing the severity or the onset of a particular condition. “Prophylactic” also includes preventing reoccurrence of a particular condition in a patient previously diagnosed with the condition. “Therapeutic” may also refer to the reduction of the severity of an existing condition. The term “treatment” is used herein to refer to any regimen that can benefit an animal, in particular a mammal, more particularly a human subject. In a particular embodiment, said mammal may be an infant or adult subject, such as a human infant or human adult.

By “cell of therapeutic interest” or “tissue of therapeutic interest”, it is meant herein a main cell or tissue where expression of the therapeutic transgene will be useful for the treatment of a disorder. In the present invention, the tissue of interest is the muscle tissue.

Hybrid Promoters

The present inventors have designed transcription regulatory elements, also referred to herein as “hybrid promoters”, for increasing gene therapy efficacy while complying with the size constraint of gene therapy vectors, such as the size constraint of AAV vectors.

The nucleic acid molecule of the invention comprises (i) one or a plurality of liver-selective enhancer(s) operably linked to (ii) a muscle-selective promoter.

The liver-selective enhancer or the plurality of liver-selective enhancer(s) may be selected from liver-selective enhancers known to those skilled in the art. In a particular embodiment, the nucleic acid molecule of the invention comprises one, and only one, liver-selective enhancer. In this embodiment, the size of the liver-selective enhancer may be from 10 to 500 nucleotides, such as from 10 to 175 nucleotides, in particular from 40 to 100 nucleotides, in particular from 50 to 80 nucleotides, more particularly from 70 to 75 nucleotides. In another embodiment, where a plurality of liver-selective enhancers is implemented, the size of the combination of the plurality of liver-selective enhancers may be from 10 to 500 nucleotides, such as from 40 to 400 nucleotides, in particular from 70 to 250 nucleotides. In a particular embodiment, the liver-selective enhancer is a naturally occurring enhancer located in cis of a gene expressed selectively in hepatocytes. In a further particular embodiment, the liver-selective enhancer may be an artificial liver-selective enhancer. Illustrative artificial liver-selective enhancers useful in the practice of the present invention include, without limitation, those disclosed in Chuah et al., Molecule Therapy, 2014, vol. 22, no. 9, p. 1605, in particular from HS-CRM1 (SEQ ID NO:21), HS-CRM2 (SEQ ID NO:22), HS-CRM3 (SEQ ID NO:23), HS-CRM4 (SEQ ID NO:24), HS-CRM5 (SEQ ID NO:25), HS-CRM6 (SEQ ID NO:26), HS-CRM7 (SEQ ID NO:27), HS-CRM8 (SEQ ID NO:1), HS-CRM9 (SEQ ID NO:28), HS-CRM10 (SEQ ID NO:29), HS-CRM11 (SEQ ID NO:30), HS-CRM12 (SEQ ID NO:31), HS-CRM13 (SEQ ID NO:32) and HS-CRM14 (SEQ ID NO:33). In a particular embodiment, the liver-selective enhancer may be selected in the group consisting of HS-CRM1, HS-CRM2, HS-CRM3, HS-CRM5, HS-CRM6, HS-CRM7, HS-CRM8, HS-CRM9, HS-CRM10, HS-CRM11, HS-CRM13 and HS-CRM14. In a further particular embodiment, the liver-selective enhancer may be selected in the group consisting of HS-CRM2, HS-CRM7, HS-CRM8, HS-CRM11, HS-CRM13 and HS-CRM14. In a particular embodiment, the liver-selective enhancer is the HS-CRM8 enhancer consisting of SEQ ID NO:1, or a functional fragment of SEQ ID NO:1 having a liver-selective enhancer activity. In another embodiment, the liver-selective enhancer is a functional variant of the HS-CRM8 enhancer that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional variant has a liver-selective enhancer activity. In a further embodiment, the liver-selective enhancer is a functional fragment of a sequence that consists of a sequence that is at least 80% identical to SEQ ID NO:1, such as at least 85% identical, in particular at least 90% identical, more particularly at least 91%, 92%, 93%, 94%, 95%, 96%, 97%, 98% or even at least 99% identical to SEQ ID NO:1, wherein said functional fragment has a liver-selective enhancer activity. In case of a plurality of liver-selective enhancers, said enhancers may be fused directly, or separated by a linker. A direct fusion means that the first nucleotide of an enhancer immediately follows the last nucleotide of upstream enhancer. In case of a link via a linker, a nucleotide sequence is present between the last nucleotide of an upstream enhancer and the first nucleotide of the following downstream enhancer. For example, the length of the linker may be comprised between 1 and 50 nucleotides, such as from 1 to 40 nucleotides, such as from 1 to 30 nucleotides, such as from 1 to 20 nucleotides, such as from 1 to 10 nucleotides. In the present invention, the design of the nucleic molecule may take into account the size constraints mentioned above and therefore, such linker(s), if any, are preferably short. Representative short linkers comprise nucleic acid sequences consisting of less than 15 nucleotides, in particular of less than 14, 13, 12, 11, 10, 9, 8, 7, 6, 5, 4, 3 or less than 2 nucleotides, such as a linker of 1 nucleotide.

The second transcription regulatory element present in the nucleic acid molecule of the invention is a muscle-selective promoter, such as a natural or synthetic muscle-selective promoter. The muscle-selective promoter is a short-sized muscle-selective promoter. In the context of the present invention, a “short-sized promoter” has a length of 2600 nucleotides or less, in particular of 2000 nucleotides or less and has a muscle-selective promoter activity when operably linked to a transgene. In a particular embodiment, the muscle-selective promoter has a length of 1500 nucleotide or less, 1100 nucleotides or less, 600 nucleotides or less, 500 nucleotides or less, 400 nucleotides or less, 300 nucleotides or less, or 200 nucleotides or less. Illustrative muscle promoters useful in the practice of the invention include, without limitation, the CK6 promoter (SEQ ID NO:6), the CK8 promoter (SEQ ID NO:7), the Actal promoter (SEQ ID NO:8) or a synthetic promoter C5.12. In a particular embodiment, the muscle-selective promoter is a synthetic promoter C5.12 (spC5.12, alternatively referred to herein as “C5.12”), such as a spC5.12 shown in SEQ ID NO:2, 3 or 4 or the spC5.12 promoter disclosed in Wang et al., Gene Therapy volume 15, pages 1489-1499 (2008). The invention may also implement functional fragments and functional variants of a muscle-selective promoter. In particular, a muscle-selective promoter useful in the practice of the present invention may be selected, without limitation, from:
a sequence that consists of the sequence shown in SEQ ID NO:2, 3, 4, 6, 7 or 8, in particular SEQ ID NO:2, 3 or 4, more particularly the sequence shown in SEQ ID NO:2, or a functional fragment of SEQ ID NO: 2, 3, 4, 6, 7 or 8, in particular SEQ ID NO:2, 3 or 4, more particularly of SEQ ID NO:2, wherein said fragment has a muscle-selective promoter activity;
a sequence that consists of a sequence that is at least 80% identical to any one of SEQ ID NO:2, 3, 4, 6, 7 or 8, such as at least 85% identical, in particular at least 90% identical, more particularly at least 91%, 92%, 93%, 94%, 95%, 96%, 97%, 98% or even at least 99% identical to any one of SEQ ID NO:2, 3, 4, 6, 7 or 8, in particular to any one of SEQ ID NO:2, 3 and 4, in particular to SEQ ID NO:2; and
a functional fragment of a sequence that consists of a sequence that is at least 80% identical to any one of SEQ ID NO:2, 3, 4, 6, 7 or 8, such as at least 85% identical, in particular at least 90% identical, more particularly at least 91%, 92%, 93%, 94%, 95%, 96%, 97%, 98% or even at least 99% identical to any one of SEQ ID NO:2, 3, 4, 6, 7 or 8, in particular to any one of SEQ ID NO:2, 3 and 4, more particularly to SEQ ID NO:2, wherein said functional fragment has a muscle-selective promoter activity. Other muscle-selective promoters include, without limitation, the MCK promoter (SEQ ID NO:14), the desmin promoter (SEQ ID NO:15) and the unc45b promoter (SEQ ID NO:16), or a functional fragment thereof having a muscle-selective promoter activity. In a further embodiment, the sequence of the muscle-selective promoter consists of a sequence that is at least 80% identical to any one of SEQ ID NO:14, 15 or 16, such as at least 85% identical, in particular at least 90% identical, more particularly at least 91%, 92%, 93%, 94%, 95%, 96%, 97%, 98% or even at least 99% identical to any one of SEQ ID NO:14, 15 or 16. In another embodiment, the muscle-selective promoter is a functional fragment of a sequence that consists of a sequence that is at least 80% identical to any one of SEQ ID NO:14, 15 or 16, such as at least 85% identical, in particular at least 90% identical, more particularly at least 91%, 92%, 93%, 94%, 95%, 96%, 97%, 98% or even at least 99% identical to any one of SEQ ID NO:14, 15 or 16, wherein said functional fragment has a muscle-selective promoter activity.

In addition, but optionally, the nucleic acid molecule described therein may further comprise a further enhancer, such as a muscle-selective enhancer, for example the MCK enhancer of SEQ ID NO:5, or a functional variant thereof having a sequence at least 80% identical to SEQ ID NO:5, such as at least 85%, 90%, 91%, 92%, 93%, 94%, 95%, 96%, 97%, 98% or even at least 99% identical to SEQ ID NO:5. In a particular embodiment, the further enhancer, in particular the MCK enhancer of SEQ ID NO:5 or a functional thereof, is located between
the liver-selective enhancer or the plurality of liver-selective enhancers; and
the muscle-selective promoter.

In the context of the present invention, the transcription regulatory elements (i.e. (i) the liver-selective enhancer or the plurality of enhancer(s); (ii) the optional other enhancer mentioned in the preceding paragraph; and (iii) the muscle-selective promoter) introduced into the nucleic acid molecule of the invention may be either fused directly or linked via a linker. For example, in case of a design with one liver-selective enhancer and a muscle-selective promoter, a direct fusion means that the first nucleotide of the promoter immediately follows the last nucleotide of the liver-selective enhancer. In addition, in case of a design with a plurality of liver-selective enhancers and a muscle-selective promoter, a direct fusion means that the first nucleotide of the promoter immediately follows the last nucleotide of the most 3′ liver-selective enhancer. In case of a link via a linker, a nucleotide sequence is present between the last nucleotide of the only liver-selective enhancer and the first nucleotide of the promoter, or between the last nucleotide of the most 3′ liver-selective enhancer and the first nucleotide of the promoter. For example, the length of the linker may be comprised between 1 and 1500 nucleotides, such as from 1 to 1000 nucleotides (e.g. 101, 300, 500 or 1000 nucleotides, such as the linkers shown in SEQ ID NO:17, 18, 19 and 20, respectively), such as from 1 and 500 nucleotides, such as from 1 and 300 nucleotides, such as from 1 and 100 nucleotides, such as from 1 to 50 nucleotides, such as from 1 to 40 nucleotides, such as from 1 to 30 nucleotides, such as from 1 to 20 nucleotides, such as from 1 to 10 nucleotides. In the present invention, the design of the nucleic molecule may take into account the size constraints mentioned above and therefore such linker(s), if any, is preferably short. Representative short linkers comprise nucleic acid sequences consisting of less than 15 nucleotides, in particular of less than 14, 13, 12, 11, 10, 9, 8, 7, 6, 5, 4, 3 or less than 2 nucleotides, such as a linker of 1 nucleotide.

In a particular embodiment, the nucleic acid molecule of the invention comprises, in particular in this order from 5′ to 3′:
one selective liver-selective, in particular the HS-CRM8 enhancer, or a functional variant or functional fragment thereof; and
a muscle-selective promoter, in particular a spC5.12 promoter or a functional variant or functional fragment thereof.

According to a particular variant of this embodiment, the nucleic acid molecule of the invention consists of the sequence shown in SEQ ID NO:9, or a functional variant thereof having a sequence at least 80% identical to SEQ ID NO:9, such as at least 85%, 90%, 91%, 92%, 93%, 94%, 95%, 96%, 97%, 98% or even at least 99% identical to SEQ ID NO:9 having a muscle-selective promoter activity.

In a particular embodiment, the nucleic acid molecule of the invention comprises, in particular in this order from 5′ to 3′:
one selective liver-selective, in particular the HS-CRM8 enhancer, or a functional variant or functional fragment thereof;
a muscle-selective enhancer such as the MCK enhancer; and
a muscle-selective promoter, in particular a spC5.12 promoter, or a functional variant or functional fragment thereof.

According to a particular variant of this embodiment, the nucleic acid molecule of the invention consists of the sequence shown in SEQ ID NO:10, or a functional variant thereof having a sequence at least 80% identical to SEQ ID NO:10, such as at least 85%, 90%, 91%, 92%, 93%, 94%, 95%, 96%, 97%, 98% or even at least 99% identical to SEQ ID NO:10 having a muscle-selective promoter activity.

In a particular embodiment, the nucleic acid molecule of the invention comprises, in particular in this order from 5′ to 3′:
two selective liver-selective enhancers, in particular two repeats of the HS-CRM8 enhancer or of a functional variant or functional fragment thereof; and
a muscle-selective promoter, in particular a spC5.12 promoter, or a functional variant or functional fragment thereof.

In a particular embodiment, the nucleic acid molecule of the invention comprises, in particular in this order from 5′ to 3′:
two selective liver-selective enhancers, in particular two repeats of the HS-CRM8 enhancer, or of a functional variant or functional fragment thereof
a muscle-selective enhancer such as the MCK enhancer; and
a muscle-selective promoter, in particular a spC5.12 promoter, or a functional variant or functional fragment thereof.

In a particular embodiment, the nucleic acid molecule of the invention comprises, in particular in this order from 5′ to 3′:
three selective liver-selective enhancers, in particular three repeats of the HS-CRM8 enhancer, or of a functional variant or functional fragment thereof; and
a muscle-selective promoter, in particular a spC5.12 promoter or a functional variant or functional fragment thereof.

According to a particular variant of this embodiment, the nucleic acid molecule of the invention consists of the sequence shown in SEQ ID NO:11, or a functional variant thereof having a sequence at least 80% identical to SEQ ID NO:11, such as at least 85%, 90%, 91%, 92%, 93%, 94%, 95%, 96%, 97%, 98% or even at least 99% identical to SEQ ID NO:11 having a muscle-selective promoter activity.

In a particular embodiment, the nucleic acid molecule of the invention comprises, in particular in this order from 5′ to 3′:
three selective liver-selective enhancers, in particular three repeats of the HS-CRM8 enhancer, or of a functional variant or functional fragment thereof
a muscle-selective enhancer such as the MCK enhancer; and
a muscle-selective promoter, in particular a spC5.12 promoter or a functional variant or functional fragment thereof.

According to a particular variant of this embodiment, the nucleic acid molecule of the invention consists of the sequence shown in SEQ ID NO:12, or a functional variant thereof having a sequence at least 80% identical to SEQ ID NO:12, such as at least 85%, 90%, 91%, 92%, 93%, 94%, 95%, 96%, 97%, 98% or even at least 99% identical to SEQ ID NO:12 having a muscle-selective promoter activity.

In all the embodiments of the nucleic acid molecule of the invention specifically disclosed herein, said nucleic acid molecule may include a linker located between a liver-selective enhancer and the muscle-selective promoter.

Furthermore, in all the embodiments of the nucleic acid molecule of the invention specifically disclosed herein, said nucleic acid molecule may include a linker located between two liver-selective enhancers. For example, in an embodiment comprising two liver-selective enhancers, a linker may be located or not between these two liver-selective enhancers. In addition, in an embodiment, comprising three liver-selective enhancers, a linker may be comprised between the first and second liver-selective enhancers and/or between the second and third liver-selective enhancers. For example, in an embodiment with three liver-selective enhancers, a linker is located between the first and second liver-selective enhancers, and no linker is located between the second and third liver-selective enhancers. In another variant, in an embodiment with three liver-selective enhancers, no linker is located between the first and second liver-selective enhancers, and a linker is located between the second and third liver-selective enhancers.

Expression Cassette

The nucleic acid molecule of the invention may be introduced into an expression cassette, designed for providing the expression of a transgene of interest into a tissue of interest.

The expression cassette of the invention thus includes the nucleic acid molecule described above, and a transgene of interest.

The expression cassette may comprise at least one further regulatory sequence capable of further controlling the expression of the therapeutic transgene of interest by decreasing or suppressing its expression in certain tissues that are not of interest, of by stabilizing the mRNA coding for the protein of interest, such as a therapeutic protein, encoded by the transgene of interest. These sequences include, for example, silencers (such as tissue-specific silencers), microRNA target sequences, introns and polyadenylation signals.

In a particular embodiment, the expression cassette of the invention comprises, in this order from 5′ to 3′:
the nucleic acid molecule of the invention;
the transgene of interest; and
a polyadenylation signal.

In a particular variant of this embodiment, an intron may be introduced between the nucleic acid molecule of the invention and the transgene of interest. As a result, the intron is located between the muscle-selective promoter included in the nucleic acid molecule as described above and the transgene of interest. Alternatively, the intron may be located within the transgene of interest. In a particular embodiment, the intron may be a SV40 intron, such as a SV40 intron consisting of SEQ ID NO:13.

Of course, from the teaching disclosed herein and the general knowledge in the fields of molecular biology and gene therapy, one skilled in the art will be able to select and adapt the enhancer number, enhancer size, promoter size, linker size, and any other element such as further enhancer(s) (e.g. a MCK enhancer or a functional variant thereof) and an intron according to the size of the transgene of interest incorporated into the expression cassette.

The transgene of interest may be any transgene as described in the “definitions” section above. In addition, specific illustrative transgenes of interest are provided in the following tables, where the transgenes are regrouped by families of neuromuscular disorders that they may treat:

Muscular dystrophies

| Gene | Protein |
|---|---|
| DMD | Dystrophin |
| EMD | Emerin |
| FHL1 | Four and a half LIM domain 1 |
| LMNA | Lamin A/C |
| SYNE1 | Spectrin repeat containing, nuclear envelope 1 (nesprin 1) |
| SYNE2 | Spectrin repeat containing, nuclear envelope 2 (nesprin 2) |
| TMEM43 | Transmembrane protein 43 |
| TOR1AIP1 | Torsin A interacting protein 1 |
| DUX4 | Double homeobox 4 |
| SMCHD1 | Structural maintenance of chromosomes flexible hinge |
| | domain |
| | containing 1 |
| PTRF | Polymerase I and transcript release factor |
| MYOT | Myotilin |
| CAV3 | Caveolin 3 |
| DNAJB6 | HSP-40 homologue, subfamily B, number 6 |
| DES | Desmin |
| TNPO3 | Transportin 3 |
| HNRNPDL | Heterogeneous nuclear ribonucleoprotein D-like |
| CAPN3 | Calpain 3 |
| DYSF | Dysferlin |
| SGCG | Gamma sarcoglycan |
| SGCA | Alpha sarcoglycan |
| SGCB | Beta sarcoglycan |
| SGCD | Delta-sarcoglycan |
| TCAP | Telethonin |
| TRIM32 | Tripartite motif-containing 32 |
| FKRP | Fukutin-related protein |
| TTN | Titin |
| POMT1 | Protein-O-mannosyltransferase 1 |
| ANO5 | Anoctamin 5 |
| FKTN | Fukutin |
| POMT2 | Protein-O-mannosyltransferase 2 |
| POMGNT1 | O-linked mannose beta1,2-N-acetylglucosaminyltransferase |
| PLEC | Plectin |
| TRAPPC11 | trafficking protein particle complex 11 |
| GMPPB | GDP-mannose pyrophosphorylase B |
| DAG1 | Dystroglycan1 |
| DPM3 | Dolichyl-phosphate mannosyltransferase polypeptide 3 |
| ISPD | Isoprenoid synthase domain containing |
| VCP | Valosin-containing protein |
| LIMS2 | LIM and senescent cell antigen-like domains 2 |
| GAA | Glucosidase alpha, acid |

Congenital muscular dystrophies

| Gene | Protein |
|---|---|
| LAMA2 | Laminin alpha 2 chain of merosin |
| COL6A1 | Alpha 1 type VI collagen |
| COL6A2 | Alpha 2 type VI collagen |
| COL6A3 | Alpha 3 type VI collagen |
| SEPN1 | Selenoprotein N1 |
| FHL1 | Four and a half LIM domain 1 |
| ITGA7 | Integrin alpha 7 precursor |
| DNM2 | Dynamin 2 |
| TCAP | Telethonin |
| LMNA | Lamin A/C |
| FKTN | Fukutin |
| POMT1 | Protein-O-mannosyltransferase 1 |
| POMT2 | Protein-O-mannosyltransferase 2 |
| FKRP | Fukutin-related protein |
| POMGNT 1 | O-linked mannose beta1,2- |
| | N-acetylglucosaminyltransferase |
| ISPD | Isoprenoid synthase domain containing |
| POMGNT2 | protein O-linked mannose |
| | N-acetylglucosaminyltransferase 2 |
| B3GNT1 | UDP-GlcNAc:betaGal beta-1,3- |
| | N-acetylglucosaminyl-transferase 1 |
| GMPPB | GDP-mannose pyrophosphorylase B |
| LARGE | Like-glycosyltransferase |
| DPM1 | Dolichyl-phosphate mannosyltransferase 1, |
| | catalytic subunit |
| DPM2 | Dolichyl-phosphate mannosyltransferase |
| | polypeptide 2, regulatory subunit |
| ALG13 | UDP-N-acetylglucosami-nyltransferase |
| B3GALNT2 | Beta-1,3-N-acetylgalacto-saminyltransferase 2 |
| TMEM5 | Transmembrane protein 5 |
| POMK | Protein-O-mannose kinase |
| CHKB | Choline kinase beta |
| ACTA1 | Alpha actin, skeletal muscle |
| TRAPPC11 | trafficking protein particle complex 11 |

Congenital myopathies

| Gene | Protein |
|---|---|
| TPM3 | Tropomyosin 3 |
| NEB | Nebulin |
| ACTA1 | Alpha actin, skeletal muscle |
| TPM2 | Tropomyosin 2 (beta) |
| TNNT1 | Slow troponin T |
| KBTBD13 | Kelch repeat and BTB (POZ) domain containing 13 |
| CFL2 | Cofilin 2 (muscle) |
| KLHL40 | Kelch-like family member 40 |
| KLHL41 | Kelch-like family member 41 |
| LMOD3 | Leiomodin 3 (fetal) |
| SEPN1 | Selenoprotein N1 |
| RYR1 | Ryanodine receptor 1 (skeletal) |
| MYH7 | Myosin, heavy polypeptide 7, cardiac muscle, beta |
| MTM1 | Myotubularin |
| DNM2 | Dynamin 2 |
| BIN1 | Amphiphysin |
| TTN | Titin |
| SPEG | SPEG complex locus |
| MEGF10 | Multiple EGF-like-domains 10 |
| MYH2 | Myosin, heavy polypeptide 2, skeletal muscle |
| MYBPC3 | Cardiac myosin binding protein-C |
| CNTN1 | Contactin-1 |
| TRIM32 | Tripartite motif-containing 32 |
| PTPLA | Protein tyrosine phosphatase-like |
| | (3-Hydroxyacyl-CoA dehydratase |
| CACNA1S | Calcium channel, voltage-dependent, |
| | L type, alpha 1S subunit |

Distal myopathies

| Gene symbol | protein |
|---|---|
| DYSF | Dysferlin |
| TTN | Titin |
| GNE | UDP-N-acetylglucosamine-2-epimerase/N- |
| | acetylmannosamine kinase |
| MYH7 | Myosin, heavy polypeptide 7, cardiac muscle, beta |
| MATR3 | Matrin 3 |
| TIA1 | Cytotoxic granuleassociated RNA binding protein |
| MYOT | Myotilin |
| NEB | Nebulin |
| CAV3 | Caveolin 3 |
| LDB3 | LIM domain binding 3 |
| ANO5 | Anoctamin 5 |
| DNM2 | Dynamin 2 |
| KLHL9 | Kelch-like homologue 9 |
| FLNC | Filamin C, gamma (actin-binding protein-280) |
| VCP | Valosin-containing protein |

Other myopathies

| Gene symbol | protein |
|---|---|
| ISCU | Iron-sulfur cluster scaffold homolog (E. coli) |
| MSTN | Myostatin |
| FHL1 | Four and a half LIM domain 1 |
| BAG3 | BCL2-associated athanogene 3 |
| ACVR1 | Activin A receptor, type II-like kinase 2 |
| MYOT | Myotilin |
| FLNC | Filamin C, gamma (actin-binding protein-280) |
| LDB3 | LIM domain binding 3 |
| LAMP2 | Lysosomal-associated membrane protein 2 precursor |
| VCP | Valosin-containing protein |
| CAV3 | Caveolin 3 |
| SEPN1 | Selenoprotein N1 |
| CRYAB | Crystallin, alpha B |
| DES | Desmin |
| VMA21 | VMA21 Vacuolar H+-ATPase Homolog (S. Cerevisiae) |
| PLEC | plectin |
| PABPN1 | Poly(A) binding protein, nuclear 1 |
| TTN | Titin |
| RYR1 | Ryanodine receptor 1 (skeletal) |
| CLN3 | Ceroid-lipofuscinosis, neuronal 3 (=battenin) |
| TRIM54 | |
| TRIM63 | Tripartite motif containing 63, E3 ubiquitin protein ligase |

Myotonic syndromes

| Gene | protein |
|---|---|
| DMPK | Myotonic dystrophy protein kinase |
| CNPB | Cellular nucleic acid-binding protein |
| CLCN1 | Chloride channel 1, skeletal muscle |
| | (Thomsen disease, autosomal dominant) |
| CAV3 | Caveolin 3 |
| HSPG2 | Perlecan |
| ATP2A1 | ATPase, Ca++ transporting, fast twitch 1 |

Ion Channel muscle diseases

| Gene | protein |
|---|---|
| CLCN1 | Chloride channel 1, skeletal muscle (Thomsen disease, |
| | autosomal dominant) |
| SCN4A | Sodium channel, voltage-gated, type IV, alpha |
| SCN5A | Voltage-gated sodium channel type V alpha |
| CACNA1S | Calcium channel, voltage-dependent, L type, |
| | alpha 1S subunit |
| CACNA1A | Calcium channel, voltage-dependent, P/Q type, alpha 1A |
| | subunit |
| KCNE3 | Potassium voltage-gated channel, Isk-related family, |
| | member 3 |
| KCNA1 | Potassium voltage-gated channel, shaker-related |
| | subfamily, member 1 |
| KCNJ18 | Kir2.6 (inwardly rectifying potassium channel 2.6) |
| KCNJ2 | Potassium inwardly-rectifying channel J2 |
| KCNH2 | Voltage-gated potassium channel, subfamily H, member 2 |
| KCNQ1 | Potassium voltage-gated channel, KQT-like subfamily, |
| | member 1 |
| KCNE2 | Potassium voltage-gated channel, Isk-related family, |
| | member 2 |
| KCNE1 | Potassium voltage-gated channel, Isk-related family, |
| | member 1 |

Malignant hyperthermia

| Gene | protein |
|---|---|
| RYR1 | Ryanodine receptor 1 (skeletal) |
| CACNA1S | Calcium channel, voltage-dependent, |
| | L type, alpha 1S subunit |

Metabolic myopathies

| Gene | protein |
|---|---|
| GAA | Acid alpha-glucosidase preproprotein |
| AGL | Amylo-1,6-glucosidase, 4-alpha-glucanotransferase |
| GBE1 | Glucan (1,4-alpha-), branching enzyme 1 (glycogen |
| | branching enzyme, Andersen disease, |
| | glycogen storage disease type IV) |
| PYGM | Glycogen phosphorylase |
| PFKM | Phosphofructokinase, muscle |
| PHKA1 | Phosphorylase b kinase, alpha submit |
| PGM1 | Phosphoglucomutase 1 |
| GYG1 | Glycogenin 1 |
| GYS1 | Glycogen synthase 3 glycogen synthase 1 (muscle) |
| | glycogen synthase 1 (muscle) |
| PRKAG2 | Protein kinase, AMP-activated, gamma 2 non-catalytic |
| | subunit |
| RBCK1 | RanBP-type and C3HC4-type zinc finger containing 1 |
| | (hemeoxidizedIRP2 ubiquitin ligase 1) |
| PGK1 | Phosphoglycerate kinase 1 |
| PGAM2 | Phosphoglycerate mutase 2 (muscle) |
| LDHA | Lactate dehydrogenase A |
| ENO3 | Enolase 3, beta muscle specific |
| CPT2 | Carnitine palmitoyltransferase II |
| SLC22A5 | Solute carrier family 22 member 5 |
| SLC25A20 | Carnitine-acylcarnitine translocase |
| ETFA | Electron-transfer-flavoprotein, alpha polypeptide |
| ETFB | Electron-transfer-flavoprotein, beta polypeptide |
| ETFDH | Electron-transferring-flavoprotein dehydrogenase |
| ACADVL | Acyl-Coenzyme A dehydrogenase, very long chain |
| ABHD5 | Abhydrolase domain containing 5 |
| PNPLA2 | Adipose triglyceride lipase (desnutrin) |
| LPIN1 | Lipin 1 (phosphatidic acid phosphatase 1) |
| PNPLA8 | Patatin-like phospholipase domain containing 8 |

Hereditary Cardiomyopathies

| Gene | protein |
|---|---|
| MYH6 | Myosin heavy chain 6 |
| MYH7 | Myosin, heavy polypeptide 7, cardiac muscle, beta |
| TNNT2 | Troponin T2, cardiac |
| TPM1 | Tropomyosin 1 (alpha) |
| MYBPC3 | Cardiac myosin binding protein-C |
| PRKAG2 | Protein kinase, AMP-activated, gamma 2 non-catalytic |
| | subunit |
| TNNI3 | Troponin I, cardiac |
| MYL3 | Myosin light chain 3 |
| TTN | Titin |
| MYL2 | Myosin light chain 2 |
| ACTC1 | Actin, alpha, cardiac muscle precursor |
| CSRP3 | Cysteine and glycine-rich protein 3 (cardiac LIM protein) |
| TNNC1 | Slow troponin C |
| VCL | Vinculin |
| MYLK2 | Myosin light chain kinase 2 |
| CAV3 | Caveolin 3 |
| MYOZ2 | Myozenin 2, or calsarcin 1, a Z disk protein |
| JPH2 | Junctophilin-2 |
| PLN | Phospholamban |
| NEXN | Nexilin(F-actin binding protein) |
| ANKRD1 | Ankyrin repeat domain 1 (cardiac muscle) |
| ACTN2 | Actinin alpha2 |
| NDUFAF1 | NADH-ubiquinone oxidoreductase 1 alpha subcomplex |
| TSFM | Ts translation elongation factor, mitochondrial |
| AARS2 | Alanyl-tRNA synthetase 2, mitochondrial |
| MRPL3 | Mitochondrial ribosomal protein L3 |
| COX15 | COX15 homolog, cytochrome c oxidase assembly protein |
| | (yeast) |
| MTO1 | Mitochondrial tRNA translation optimization 1 |
| MRPL44 | Mitochondrial ribosomal protein L44 |
| LMNA | Lamin A/C |
| LDB3 | LIM domain binding 3 |
| SCN5A | Voltage-gated sodium channel type V alpha |
| DES | Desmin |
| EYA4 | Eyes absent 4 |
| SGCD | Delta-sarcoglycan |
| TCAP | Telethonin |
| ABCC9 | ATP-binding cassette, sub-family C (member 9) |
| TMPO | Lamina-associated polypeptide 2 |
| PSEN2 | Presenilin 2 |
| CRYAB | Crystallin, alpha B |
| FKTN | Fukutin |
| TAZ | Tafazzin |
| DMD | Dystrophin |
| LAMA4 | Laminin alpha 4 |
| ILK | Integrin-linked kinase |
| MYPN | Myopalladin |
| RBM20 | RNA binding motif protein 20 |
| SYNE1 | Spectrin repeat containing, nuclear envelope 1 (nesprin 1) |
| MURC | Muscle-related coiled-coil protein |
| DOLK | Dolichol kinase |
| GATAD1 | GATA zinc finger domain containing 1 |
| SDHA | succinate dehydrogenase complex, subunit A, flavoprotein |
| | (Fp) |
| GAA | Acid alpha-glucosidase preproprotein |
| DTNA | Dystrobrevin, alpha |
| FLNA | Filamin A, alpha (actin binding protein 280) |
| TGFB3 | Transforming growth factor, beta 3 |
| RYR2 | Ryanodine receptor 2 |
| TMEM43 | Transmembrane protein 43 |
| DSP | Desmoplakin |
| PKP2 | Plakophilin 2 |
| DSG2 | Desmoglein 2 |
| DSC2 | Desmocollin 2 |
| JUP | Junction plakoglobin |
| CASQ2 | Calsequestrin 2 (cardiac muscle) |
| KCNQ1 | Potassium voltage-gated channel, KQT-like subfamily, |
| | member 1 |
| KCNH2 | Voltage-gated potassium channel, subfamily H, member 2 |
| ANK2 | Ankyrin 2 |
| KCNE1 | Potassium voltage-gated channel, Isk-related family, |
| | member 1 |
| KCNE2 | Potassium voltage-gated channel, Isk-related family, |
| | member 2 |
| KCNJ2 | Potassium inwardly-rectifying channel J2 |
| CACNA1C | Calcium channel, voltage-dependent, L type, alpha 1C |
| | subunit |
| SCN4B | Sodium channel, voltage-gated, type IV, beta subunit |
| AKAP9 | A kinase (PRKA) anchor protein (yotiao) 9 |
| SNTA1 | Syntrophin, alpha 1 |
| KCNJ5 | Potassium inwardly-rectifying channel, subfamily J, |
| | member 5 |
| NPPA | Natriuretic peptide precursor A |
| KCNA5 | Potassium voltage-gated channel, shaker-related sub- |
| | family, member 5 |
| GJA5 | Connexin 40 |
| SCN1B | Sodium channel, voltage-gated, type I, beta subunit |
| SCN2B | Sodium channel, voltage-gated, type II, beta subunit |
| NUP155 | Nucleoporin 155 kDa |
| GPD1L | Glycerol-3-phosphate dehydrogenase 1-like |
| CACNB2 | Calcium channel, voltage-dependent, beta 2 subunit |
| KCNE3 | Potassium voltage-gated channel, Isk-related family, |
| | member 3 |
| SCN3B | Sodium channel, voltage-gated, type III, beta subunit |
| HCN4 | Hyperpolarization activated cyclic nucleotide-gated |
| | potassium channel 4 |

Congenital myasthenic syndromes

| Gene | protein |
|---|---|
| CHRNA1 | Cholinergic receptor, nicotinic, alpha polypeptide 1 |
| CHRNB1 | Cholinergic receptor, nicotinic, beta 1 muscle |
| CHRND | Cholinergic receptor, nicotinic, delta |
| CHRNE | Cholinergic receptor, nicotinic, epsilon |
| RAPSN | Rapsyn |
| CHAT | Choline acetyltransferase isoform |
| COLQ | Acetylcholinesterase collagen-like tail subunit |
| MUSK | muscle, skeletal, receptor tyrosine kinase |
| DOK7 | Docking protein 7 |
| AGRN | Agrin |
| GFPT1 | Glutamine-fructose-6-phosphate transaminase 1 |
| DPAGT1 | Dolichyl-phosphate (UDP-N-acetylglucosamine) N- |
| | acetylglucosaminephosphotransferase 1 |
| | (GlcNAc-1-P transferase) |
| LAMB2 | Laminin, beta 2 (laminin S) |
| SCN4A | Sodium channel, voltage-gated, type IV, alpha |
| CHRNG | Cholinergic receptor, nicotinic, gamma polypeptide |
| PLEC | plectin |
| ALG2 | Alpha-1,3/1,6-mannosyltransferase |
| ALG14 | UDP-N-acetylglucosaminyltransferase |
| SYT2 | Synaptotagmin II |
| PREPL | Prolyl endopeptidase-like |

Motor Neuron diseases

| Gene | protein |
|---|---|
| SMN1 | Survival of motor neuron 1, telomeric |
| IGHMBP2 | Immunoglobulin mu binding protein 2 |
| PLEKHG5 | Pleckstrin homology domain containing, family G (with |
| | RhoGef domain) member 5 |
| HSPB8 | Heat shock 27 kDa protein 8 |
| HSPB1 | Heat shock 27 kDa protein 1 |
| HSPB3 | Heat shock 27 kDa protein 3 |
| AARS | Alanyl-tRNA synthetase |
| GARS | Glycyl-tRNA synthetase |
| BSCL2 | Seipin |
| REEP1 | Receptor accessory protein 1 |
| SLC5A7 | Solute carrier family 5 (sodium/choline cotransporter), |
| | member 7 |
| DCTN1 | Dynactin 1 |
| UBA1 | Ubiquitin-activating enzyme 1 |
| ATP7A | ATPase, Cu++ transporting, alpha polypeptide |
| DNAJB2 | DnaJ (Hsp40) homolog, subfamily B, member 2 |
| TRPV4 | Transient receptor potential cation channel, subfamily V, |
| | member 4 |
| DYNC1H1 | Dynein, cytoplasmic 1, heavy chain 1 |
| BICD2 | Bicaudal D homolog 2 (Drosophila) |
| FBXO38 | F-box protein 38 |
| ASAH1 | N-acylsphingosine amidohydrolase (acid ceramidase) 1 |
| VAPB | Vesicle-associated membrane protein-associated protein B |
| | and C |
| EXOSC8 | Exosome component 8 |
| SOD1 | Superoxide dismutase 1, soluble |
| ALS2 | Alsin |
| SETX | Senataxin |
| FUS | Fusion (involved in t(12;16) in malignant liposarcoma) |
| ANG | Angiogenin |
| TARDBP | TAR DNA binding protein |
| FIG4 | Sac domain-containing inositol phosphatase 3 |
| OPTN | Optineurin |
| ATXN2 | Ataxin 2 |
| VCP | Valosin-containing protein |
| UBQLN2 | Ubiquilin 2 |
| SIGMAR1 | Sigma non-opioid intracellular receptor 1 |
| CHMP2B | Charged multivesicular body protein 2B |
| PFN1 | Profilin 1 |
| MATR3 | Matrin 3 |
| NEFH | Neurofilament, heavy polypeptide |
| PRPH | Peripherin |
| C9orf72 | Chromosome 9 open reading frame 72 |
| CHCHD10 | Coiled-coil-helix-coiled-coil-helix domain containing 10 |
| SQSTM1 | Sequestosome 1 |
| AR | Androgen receptor |
| GLE1 | GLE1 RNA export mediator homolog (yeast) |
| ERBB3 | V-erb-b2 erythroblastic leukemia viral oncogene |
| | homolog 3 (avian) |
| PIP5K1C | Phosphatidylinositol-4-phosphate 5-kinase, type I, gamma |
| EXOSC3 | Exosome component 3 |
| VRK1 | Vaccinia related kinase 1 |
| SLC52A3 | Solute carrier family 52, riboflavin transporter, member 3 |
| SLC52A2 | Solute carrier family 52, riboflavin transporter, member 2 |
| HEXB | Hexosaminidase B |

Hereditary motor and sensory neuropathies

| Gene | Protein |
|---|---|
| PMP22 | Peripheral myelin protein 22 |
| MPZ | Myelin protein zero |
| LITAF | Lipopolysaccharide-induced TNF factor |
| EGR2 | Early growth response 2 protein |
| NEFL | Neurofilament, light polypeptide 68 kDa |
| HOXD10 | Homeobox D10 |
| ARHGEF10 | Rho guanine nucleotide exchange factor 10 |
| FBLN5 | Fibulin 5 (extra-cellular matrix) |
| DNM2 | Dynamin 2 |
| YARS | Tyrosyl-tRNA synthetase |
| INF2 | Inverted formin 2 |
| GNB4 | Guanine nucleotidebinding protein, beta-4 |
| GDAP1 | Ganglioside-induced differentiation-associated protein 1 |
| MTMR2 | Myotubularin-related protein 2 |
| SBF2 | SET binding factor 2 |
| SBF1 | SET binding factor 1 |
| SH3TC2 | KIAA1985 protein |
| NDRG1 | N-myc downstream regulated gene 1 |
| PRX | Periaxin |
| HK1 | Hexokinase 1 |
| FGD4 | Actin-filament binding protein Frabin |
| FIG4 | Sac domain-containing inositol phosphatase 3 |
| SURF1 | surfeit 1 |
| GJB1 | Gap junction protein, beta 1, 32 kDa (connexin 32) |
| AIFM1 | Apoptosis-inducing factor, mitochondrionassociated 1 |
| PRPS1 | Phosphoribosyl pyrophosphate synthetase 1 |
| PDK3 | Pyruvate dehydrogenase kinase, isoenzyme 3 |
| KIF1B | Kinesin family member 1B |
| MFN2 | Mitofusin 2 |
| RAB7A | RAB7, member RAS oncogene family |
| TRPV4 | Transient receptor potential cation channel, subfamily V, |
| | member 4 |
| GARS | Glycyl-tRNA synthetase |
| HSPB1 | Heat shock 27 kDa protein 1 |
| HSPB8 | Heat shock 27 kDa protein 8 |
| AARS | Alanyl-tRNA synthetase |
| DYNC1H1 | Dynein, cytoplasmic 1, heavy chain 1 |
| LRSAM1 | leucine rich repeat and sterile alpha motif containing 1 |
| DHTKD1 | dehydrogenase E1 and transketolase domain containing 1 |
| TRIM2 | Tripartite motif containing 2 |
| TFG | TRK-fused gene |
| MARS | methionyl-tRNA synthetase |
| KIF5A | Kinesin family member 5A |
| LMNA | Lamin A/C |
| MED25 | Mediator complex subunit 25 |
| DNAJB2 | DnaJ (Hsp40) homolog, subfamily B, member 2 |
| HINT1 | Histidine triad nucleotide binding protein 1 |
| KARS | Lysyl-tRNA synthetase |
| PLEKHG5 | Pleckstrin homology domain containing, family G |
| | (with RhoGef domain) member 5 |
| COX6A1 | Cytochrome c oxidase subunit VIa polypeptide 1 |
| IGHMBP2 | Immunoglobulin mu binding protein 2 |
| SPTLC1 | Serine palmitoyltransferase subunit 1 |
| SPTLC2 | Serine palmitoyltransferase long chain base subunit 2 |
| ATL1 | Atlastin GTPase 1 |
| KIF1A | Kinesin family member 1A |
| WNK1 | WNK lysine deficient protein kinase 1 |
| IKBKAP | Inhibitor of kappa light polypeptide gene enhancer in |
| | B-cells, kinase complex-associated protein |
| NGF | Nerve growth factor (beta polypeptide) |
| DNMT1 | DNA (cytosine-5)-methyltransferase 1 |
| SLC12A6 | Potassium chloride cotransporter KCC3 |
| GJB3 | Gap junction protein, beta 3, 31 kDa (=connexin 31) |
| sept-09 | Septin 9 |
| GAN | Gigaxonin |
| CTDP1 | CTD phosphatase subunit 1 |
| VRK1 | Vaccinia related kinase 1 |

Hereditary paraplegia

| Gene symbol | protein |
|---|---|
| ATL 1 | Atlastin |
| SPAST | Spastin |
| NIPA1 | Non-imprinted in Prader-Willi/Angelman syndrome 1 |
| KIAA0196 | Strumpellin |
| KIF5A | Kinesin family member 5A |
| RTN2 | Reticulon 2 |
| HSPD1 | Heat shock 60 kDa protein 1 (chaperonin) |
| BSCL2 | Seipin |
| REEP1 | Receptor accessory protein 1 |
| ZFYVE27 | Protrudin |
| SLC33A1 | Solute carrier family 33 (acetyl- CoA transporter) |
| CYP7B1 | Cytochrome P450, family 7, subfamily B, polypeptide 1 |
| SPG7 | Paraplegin |
| SPG11 | Spatacsin |
| ZFYVE26 | Spastizin |
| ERLIN2 | ER lipid raft associated 2 |
| SPG20 | Spartin |
| SPG21 | Maspardin |
| B4GALNT1 | beta-1,4-N-acetyl-galactosaminyl transferase 1 |
| DDHD1 | DDHD domain containing 1 |
| KIF1A | Kinesin family member 1A |
| FA2H | Fatty acid 2-hydroxylase |
| PNPLA6 | Patatin-like phospholipase domain containing 6 |
| C19orf12 | chromosome 19 open reading frame 12 |
| GJC2 | gap junction protein, gamma 2, 47 kDa |
| NT5C2 | 5′-nucleotidase, cytosolic II |
| GBA2 | glucosidase, beta (bile acid) 2 |
| AP4B1 | adaptor-related protein complex 4, beta 1 subunit |
| AP5Z1 | Hypothetical protein LOC9907 |
| TECPR2 | tectonin beta-propeller repeat containing 2 |
| AP4M1 | Adaptor-related protein complex 4, mu 1 subunit |
| AP4E1 | Adaptor-related protein complex 5, zeta 1 subunit |
| AP4S1 | adaptor-related protein complex 4, sigma 1 subunit |
| DDHD2 | DDHD domain containing 2 |
| C12orf65 | adaptor-related protein complex 4, sigma 1 subunit |
| CYP2U1 | cytochrome P450, family 2, subfamily U, polypeptide 1 |
| ARL6IP1 | ADP-ribosylation factor-like 6 interacting protein 1 |
| AMPD2 | adenosine monophosphate deaminase 2 |
| ENTPD1 | ectonucleoside triphosphate diphosphohydrolase 1 |
| ALDH3A2 | Aldehyde dehydrogenase 3A2 |
| ALS2 | Alsin |
| L1CAM | L1 cell adhesion molecule |
| PLP1 | Proteolipid protein 1 |
| MTPAP | mitochondrial poly(A) polymerase |
| AFG3L2 | AFG3 ATPase family gene 3-like 2 (S. cerevisiae) 1 |
| SACS | Sacsin |

Other neuromuscular disorders

| Gene | protein |
|---|---|
| TOR1A | Torsin A |
| SGCE | Sarcoglycan, epsilon |
| IKBKAP | Inhibitor of kappa light polypeptide gene enhancer in |
| | B-cells, kinase complex-associated protein |
| TTR | Transthyretin (prealbumin, amyloidosis type I) |
| KIF21A | Kinesin family member 21A |
| PHOX2A | Paired-like aristaless homeobox protein 2A |
| TUBB3 | Tubulin, beta 3 |
| TPM2 | Tropomyosin 2 (beta) |
| MYH3 | Myosine, heavy chain 3, skeletal muscle, embryonic |
| TNNI2 | Troponin I, type 2 |
| TNNT3 | Troponin T3, skeletal |
| SYNE1 | Spectrin repeat containing, nuclear envelope 1 (nesprin 1) |
| MYH8 | Myosin heavy chain, 8, skeletal muscle, perinatal |
| POLG | Polymerase (DNA directed), gamma |
| SLC25A4 | Mitochondrial carrier; adenine nucleotide translocator |
| C10orf2 | chromosome 10 open reading frame 2 |
| POLG2 | Mitochondrial DNA polymerase, accessory subunit |
| RRM2B | Ribonucleotide reductase M2 B (TP53 inducible) |
| TK2 | Thymidine kinase 2, mitochondrial |
| SUCLA2 | Succinate-CoA ligase, ADP-forming, beta subunit |
| OPA1 | optic atrophy 1 |
| STIM1 | Stromal interaction molecule 1 |
| ORAI1 | ORAI calcium release-activated calcium modulator 1 |
| PUS1 | Pseudouridylate synthase 1 |
| CHCHD10 | Coiled-coil-helix-coiled-coil-helix domain containing 10 |
| CASQ1 | Calsequestrin 1 (fast-twitch, skeletal muscle) |
| YARS2 | tyrosyl-tRNA synthetase 2, mitochondrial |

Vectors, Cells and Pharmaceutical Compositions

The expression cassette of the invention may be introduced into a vector. Thus, the invention also relates to a vector comprising the expression cassette described above. The vector used in the present invention is a vector suitable for RNA/protein expression, and in particular suitable for gene therapy.

In one embodiment, the vector is a plasmid vector.

In another embodiment, the vector is a non-viral vector, such as a nanoparticle, a lipid nanoparticle (LNP) or a liposome, containing the expression cassette of the invention.

In another embodiment, the vector is a system based on transposons, allowing integration of the expression cassette of the invention in the genome of the target cell, such as the hyperactive Sleeping Beauty (SB100×) transposon system (Mates et al. 2009).

In a further embodiment, the transgene of interest is a repair matrix useful for targeted genome engineering, such as a repair matrix suitable for the correction of a gene along with an endonuclease as described above. More particularly, the vector includes a repair matrix containing arms of homology to a gene of interest, for homology driven integration.

In another embodiment, the vector is a viral vector suitable for gene therapy, targeting muscles. In this case, the further sequences are added to the expression cassette of the invention, suitable for producing an efficient viral vector, as is well known in the art. In a particular embodiment, the viral vector is derived from an integrating virus. In particular, the viral vector may be derived from an adenovirus, a retrovirus or a lentivirus (such as an integration-deficient lentivirus). In a particular embodiment, the lentivirus is a pseudotyped lentivirus having an enveloped that enable the targeting of cells/tissues of interest, such as liver and/or muscle cells (as described in patent applications EP17306448.6 and EP17306447.8). In case the viral vector is derived from a retrovirus or lentivirus, the further sequences are retroviral or lentiviral LTR sequences flanking the expression cassette. In another particular embodiment, the viral vector is a parvovirus vector, such as an AAV vector, such as an AAV vector suitable for transducing a muscles. In this embodiment, the further sequences are AAV ITR sequences flanking the expression cassette.

In a preferred embodiment, the vector is an AAV vector. The human parvovirus Adeno-Associated Virus (AAV) is a dependovirus that is naturally defective for replication which is able to integrate into the genome of the infected cell to establish a latent infection. The last property appears to be unique among mammalian viruses because the integration occurs at a specific site in the human genome, called AAVS1, located on chromosome 19 (19q13.3-qter). Therefore, AAV vectors have arisen considerable interest as potential vectors for human gene therapy. Among the favorable properties of the virus are its lack of association with any human disease, its ability to infect both dividing and non-dividing cells, and the wide range of cell lines derived from different tissues that can be infected.

Among the serotypes of AAVs isolated from human or non-human primates (NHP) and well characterized, human serotype 2 is the first AAV that was developed as a gene transfer vector. Other currently used AAV serotypes include AAV-1, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods.), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as cy10 and -rh10, -rh74, -rh74-9 as disclosed in EP18305399 (such as the Hybrid Cap rh74-9 serotype described in examples of EP18305399; a rh74-9 serotype being also referred to herein as “-rh74-9”, “AAVrh74-9” or “AAV-rh74-9”), -9-rh74 as disclosed in EP18305399 (such as the Hybrid Cap 9-rh74 serotype described in the examples of EP18305399; a -9-rh74 serotype being also referred to herein as “−9-rh74”, “AAV9-rh74”, “AAV-9-rh74”, or “rh74-AAV9”), -dj, Anc80, LK03, AAV2i8, porcine AAV serotypes such as AAVpo4 and AAVpo6, and tyrosine, lysine and serine capsid mutants of the AAV serotypes, etc. In addition, other non-natural engineered variants and chimeric AAV can also be useful. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells.

AAV-based recombinant vectors lacking the Rep protein integrate with low efficacy into the host's genome and are mainly present as stable circular episomes that can persist for years in the target cells.

Alternatively to using AAV natural serotypes, artificial AAV serotypes may be used in the context of the present invention, including, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid.

In the context of the present invention, the AAV vector comprises an AAV capsid able to transduce the target cells of interest, i.e. muscle cells.

According to a particular embodiment, the AAV vector is of the AAV-1, -2, AAV-2 variants (such as the quadruple-mutant capsid optimized AAV-2 comprising an engineered capsid with Y44+500+730F+T491V changes, disclosed in Ling et al., 2016 Jul. 18, Hum Gene Ther Methods. [Epub ahead of print]), -3 and AAV-3 variants (such as the AAV3-ST variant comprising an engineered AAV3 capsid with two amino acid changes, S663V+T492V, disclosed in Vercauteren et al., 2016, Mol. Ther. Vol. 24(6), p. 1042), -3B and AAV-3B variants, -4, -5, -6 and AAV-6 variants (such as the AAV6 variant comprising the triply mutated AAV6 capsid Y731F/Y705F/T492V form disclosed in Rosario et al., 2016, Mol Ther Methods Clin Dev. 3, p. 16026), -7, -8, -9, -2G9, -10 such as -cy10 and -rh10, -rh39, -rh43, -rh74, -rh74-9, -dj, Anc80, LK03, AAV.PHP, AAV2i8, porcine AAV such as AAVpo4 and AAVpo6, and tyrosine, lysine and serine capsid mutants of AAV serotypes. In a particular embodiment, the AAV vector is of the AAV8, AAV9, AAVrh74, AAVrh74-9, or AAV2i8 serotype (i.e. the AAV vector has a capsid of the AAV8, AAV9, AAVrh74, AAVrh74-9 or AAV2i8 serotype). In a further particular embodiment, the AAV vector is a pseudotyped vector, i.e. its genome and capsid are derived from AAVs of different serotypes. For example, the pseudotyped AAV vector may be a vector whose genome is derived from one of the above mentioned AAV serotypes, and whose capsid is derived from another serotype. For example, the genome of the pseudotyped vector may have a capsid derived from the AAV8, AAV9, AAVrh74, AAVrh74-9, or AAV2i8 serotype, and its genome may be derived from and different serotype. In a particular embodiment, the AAV vector has a capsid of the AAV8, AAV9, AAVrh74 or AAVrh74-9 serotype, in particular of the AAV8 or AAV9 serotype, more particularly of the AAV8 serotype.

In another embodiment, the capsid is a modified capsid. In the context of the present invention, a “modified capsid” may be a chimeric capsid or capsid comprising one or more variant VP capsid proteins derived from one or more wild-type AAV VP capsid proteins.

In a particular embodiment, the AAV vector is a chimeric vector, i.e. its capsid comprises VP capsid proteins derived from at least two different AAV serotypes, or comprises at least one chimeric VP protein combining VP protein regions or domains derived from at least two AAV serotypes. For example, a chimeric AAV vector can derive from the combination of an AAV8 capsid sequence with a sequence of an AAV serotype different from the AAV8 serotype, such as any of those specifically mentioned above.

In another embodiment, the modified capsid can be derived also from capsid modifications inserted by error prone PCR and/or peptide insertion (e.g. as described in Bartel et al., 2011). In addition, capsid variants may include single amino acid changes such as tyrosine mutants (e.g. as described in Zhong et al., 2008)

In addition, the genome of the AAV vector may either be a single stranded or self-complementary double-stranded genome (McCarty et al., Gene Therapy, 2003). Self-complementary double-stranded AAV vectors are generated by deleting the terminal resolution site from one of the AAV terminal repeats. These modified vectors, whose replicating genome is half the length of the wild type AAV genome have the tendency to package DNA dimers. In a preferred embodiment, the AAV vector implemented in the practice of the present invention has a single stranded genome, and further preferably comprises an AAV8, AAV9, AAVrh74, AAVrh74-9, or AAV2i8 capsid, in particular an AAV8, AAV9, AAVrh74 or AAVrh74-9 capsid, such as an AAV8 or AAV9 capsid, more particularly an AAV8 capsid. As is known in the art, additional suitable sequences may be introduced in the nucleic acid construct of the invention for obtaining a functional viral vector. Suitable sequences include AAV ITRs.

Of course, in designing the nucleic acid sequence of the invention and the expression cassette of the invention one skilled in the art will take care of respecting the size limit of the vector used for delivering said construct to a cell or organ. In particular, as reminded above, in case of the vector being an AAV vector one skilled in the art knows that a major limitation of AAV vector is its cargo capacity which may vary from one AAV serotype to another but is thought to be limited to around the size of parental viral genome. For example, 5 kb is the maximum size usually thought to be packaged into an AAV8 capsid. (Wu Z. et al., Mol Ther., 2010, 18(1): 80-86; Lai Y. et al., Mol Ther., 2010, 18(1): 75-79; Wang Y. et al., Hum Gene Ther Methods, 2012, 23(4): 225-33). Accordingly, those skilled in the art will take care in practicing the present invention to select the components of the nucleic acid construct of the invention so that the resulting nucleic acid sequence, including sequences coding AAV 5′- and 3′-ITRs to preferably not exceed 110% of the cargo capacity of the AAV vector implemented, in particular to preferably not exceed 5.5 kb.

The invention also relates to an isolated cell, for example muscle cell, which is transformed with a nucleic acid sequence of the invention or with the expression cassette of the invention. Cells of the invention may be delivered to the subject in need thereof via injection in the tissue of interest or in the bloodstream of said subject. In a particular embodiment, the invention involves introducing the nucleic acid molecule or the expression cassette of the invention into cells of the subject to be treated, and administering back to the subject said cells into which the nucleic acid or expression cassette has been introduced.

The present invention also provides a pharmaceutical composition comprising a nucleic acid molecule, a vector or a cell of the invention. Such compositions comprise a therapeutically effective amount of the nucleic acid sequence, vector or cell of the invention, and a pharmaceutically acceptable carrier. The term “pharmaceutically acceptable” means approved by a regulatory agency of the Federal or a state government or listed in the U.S. or European Pharmacopeia or other generally recognized pharmacopeia for use in animals, and humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, lactose, sucrose, sodium stearate, glycerol monostearate, talc, sodium chloride, dried skim milk, glycerol, propylene glycol, water, ethanol and the like.

The composition, if desired, can also contain minor amounts of wetting or emulsifying agents, or pH buffering agents. These compositions can take the form of solutions, suspensions, emulsions, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a therapeutically effective amount of the therapeutic, preferably in purified form, together with a suitable amount of carrier so as to provide the form for proper administration to the subject. In a particular embodiment, the nucleic acid sequence, expression cassette, vector or cell of the invention is formulated in a composition comprising phosphate-buffered saline and supplemented with 0.25% human serum albumin. In another particular embodiment, the vector of the invention is formulated in a composition comprising ringer lactate and a non-ionic surfactant, such as pluronic F68 at a final concentration of 0.01-0.0001%, such as at a concentration of 0.001%, by weight of the total composition. The formulation may further comprise serum albumin, in particular human serum albumin, such as human serum albumin at 0.25%. Other appropriate formulations for either storage or administration are known in the art, in particular from WO 2005/118792 or Allay et al., 2011.

In a preferred embodiment, the composition is formulated in accordance with routine procedures as a pharmaceutical composition adapted for intravenous or intramuscular administration, preferably intravenous administration, to human beings. Typically, compositions for intravenous administration are solutions in sterile isotonic aqueous buffer. Where necessary, the composition may also include a solubilizing agent and a local anesthetic such as lignocaine to, ease pain at the, site of the injection.

In an embodiment, the nucleic acid sequence, expression cassette or vector of the invention can be delivered in a vesicle, in particular a liposome. In yet another embodiment, the nucleic acid sequence, expression cassette or the vector of the invention can be delivered in a controlled release system.

Methods of Use of the Vector

Thanks to the present invention, a transgene of interest may be expressed in muscles or muscle cells.

The nucleic acid molecule, expression cassette or vector of the present invention may be used for expressing a gene into a muscle cell. Accordingly, the invention provides a method for expressing a transgene of interest in a muscle cell, wherein the expression cassette of the invention is introduced in the muscle cell, and the transgene of interest is expressed. The method may be an in vitro, ex vivo or in vivo method for expressing a transgene of interest in a muscle cell.

The nucleic acid molecule, expression cassette or vector of the present invention may also be used for gene therapy. Accordingly, in one aspect, the invention relates to a nucleic acid molecule, expression cassette, vector, cell or pharmaceutical composition as described above, for use as a medicament. In an aspect, the invention thus relates to the nucleic acid molecule, expression cassette or vector disclosed herein for use in therapy, specifically in gene therapy. Likewise, the cell of the invention may be used in therapy, specifically in cell therapy.

In another aspect, the invention relates to a nucleic acid molecule, expression cassette, vector, cell or pharmaceutical composition as described above, for use in a method for the treatment of a neuromuscular disorder.

In a further aspect, the invention relates to the use of a nucleic acid molecule, expression cassette, vector, cell or pharmaceutical composition as described above, for the manufacture of a medicament for use in the treatment of a neuromuscular disorder.

In another aspect, the invention relates to a method for the treatment of a neuromuscular disorder, comprising administering a therapeutically effective amount of the nucleic acid molecule, expression cassette, vector, cell or pharmaceutical composition described herein to a subject in need thereof.

The neuromuscular disorder is in particular an inherited or acquired disorder, such as an inherited or acquired neuromuscular disease. Of course, the therapeutic transgene and the promoter driving expression into a tissue of therapeutic interest will be selected in view of the disorder to be treated.

The term “neuromuscular disorder” encompasses diseases and ailments that impair the functioning of the muscles, either directly, being pathologies of the voluntary muscle, or indirectly, being pathologies of nerves or neuromuscular junctions. Illustrative neuromuscular disorders include, without limitation, muscular dystrophies (e.g. myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy), motor neuron diseases (e.g. amyotrophic lateral sclerosis (ALS), spinal muscular atrophy (Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4)), spinal-bulbar muscular atrophy (Kennedy disease)), inflammatory Myopathies (e.g. polymyositis dermatomyositis, inclusion-body myositis), diseases of neuromuscular junction (e.g. myasthenia gravis, Lambert-Eaton (myasthenic) syndrome, congenital myasthenic syndromes), diseases of peripheral nerve (e.g. Charcot-Marie-Tooth disease, Friedreich's ataxia, Dejerine-Sottas disease), metabolic diseases of muscle (e.g. phosphorylase deficiency (McArdle disease) acid maltase deficiency (Pompe disease) phosphofructokinase deficiency (Tarui disease) debrancher enzyme deficiency (Cori or Forbes disease) mitochondrial myopathy, carnitine deficiency, carnitine palmityl transferase deficiency, phosphogly cerate kinase deficiency, phosphoglycerate mutase deficiency, lactate dehydrogenase deficiency, myoadenylate deaminase deficiency), myopathies due to endocrine abnormalities (e.g. hyperthyroid myopathy, hypothyroid myopathy), and other myopathies (e.g. myotonia congenital, paramyotonia congenital, central core disease, nemaline myopathy, myotubular myopathy, periodic paralysis). In this embodiment, the nucleic acid sequence of the invention comprises liver-selective, muscle-selective and/or neuron-selective transcription regulatory elements, such as liver-selective and muscle-selective transcription regulatory elements, liver-selective and neuron-selective transcription regulatory elements, and liver-selective, muscle-selective and neuron-selective transcription regulatory elements

In a particular embodiment, the disorder is a glycogen storage disease. The expression “glycogen storage disease” denotes a group of inherited metabolic disorders involving enzymes responsible for the synthesis and degradation of glycogen. In a more particular embodiment, the glycogen storage disease may be GSDI (von Gierke's disease), GSDII (Pompe disease), GSDIII (Cori disease), GSDIV, GSDV, GSDVI, GSDVII, GSDVIII or lethal congenital glycogen storage disease of the heart. More particularly, the glycogen storage disease is selected in the group consisting of GSDI, GSDII and GSDIII, even more particularly in the group consisting of GSDII and GSDIII. In an even more particular embodiment, the glycogen storage disease is GSDII. In particular, the nucleic acid molecules of the invention may be useful in gene therapy to treat GAA-deficient conditions, or other conditions associated by accumulation of glycogen such as GSDI (von Gierke's disease), GSDII (Pompe disease), GSDIII (Cori disease), GSDIV, GSDV, GSDVI, GSDVII, GSDVIII and lethal congenital glycogen storage disease of the heart, more particularly GSDI, GSDII or GSDIII, even more particularly GSDII and GSDIII. In a further particular embodiment, the disorder is Pompe disease and the therapeutic transgene is a gene encoding an acid alpha-glucosidase (GAA) or a variant thereof. Such variants of GAA are in particular disclosed in applications PCT/2017/072942, PCT/EP2017/072945 and PCT/EP2017/072944, which are incorporated herein by reference in their entirety. In this embodiment, the nucleic acid sequence of the invention comprises liver-selective, muscle-selective and/or neuron-selective transcription regulatory elements, such as liver-selective and muscle-selective transcription regulatory elements, liver-selective and neuron-selective transcription regulatory elements, muscle-selective and neuron-selective transcription regulatory elements, and liver-selective, muscle-selective and neuron-selective transcription regulatory elements. In a particular embodiment, the disorder is infantile-onset Pompe disease (IOPD) or late onset Pompe disease (LOPD). Preferably, the disorder is IOPD.

One skilled in the art is aware of the transgene of interest useful in the treatment of these and other disorders by gene therapy. For example, the therapeutic transgene is: lysosomal enzymes α-L-iduronidase [IDUA (alphase—Liduronidase)], for MPSI, acid-α-glucosidase (GAA) for Pompe disease, Glycogen Debranching Enzyme (GDE) or shortened forms of GDE (also referred to as truncated forms of GDE, or mini-GDE) for Cori disease (GSDIII), G6P for GSDI, alpha-sarcoglycan (SGCA) for LGMD2D; dystrophin or its shortened forms for DMD; and SMN1 for SMA. The transgene of interest may also be a transgene that provides other therapeutic properties than providing a missing protein or a RNA suppressing the expression of a given protein. For example, transgenes of interest may include, without limitation, transgenes that may increase muscle strength.

Specific examples of therapeutic transgenes of interest that may be operably linked to the hybrid promoter of the invention for specific diseases are provided below.

In a particular embodiment, the disease is Cori disease and the transgene of interest encodes a GDE or a shortened form of GDE. Shortened forms of GDE suitable for use in the present invention may include, without limitation, those described in EP18306088. Alternatively, the present invention is used in a dual AAV vector system for expressing GDE, such as the dual AAV vector system disclosed in WO2018162748. In this embodiment, the vector of the present invention may correspond to the first AAV vector of the dual AAV vector system, comprising between 5′ and 3′ AAV ITRs, a first nucleic acid sequence that encodes a N-terminal part of a GDE under the control of a nucleic acid molecule of the present invention.

In another particular embodiment, the disease is Pompe disease, and the transgene of interest encodes an acid-α-glucosidase (GAA), or a modified GAA. Modified GDE suitable for use in the present invention include, without limitation, those disclosed in WO2018046772, WO2018046774 and WO2018046775.

In a further particular embodiment, the disorder is selected from Duchene muscular dystrophy, myotubular myopathy, spinal muscular atrophy, limb-girdle muscular dystrophy type 21, 2A, 2B, 2C or 2D and myotonic dystrophy type 1.

Methods of administration of the vector of the invention include but are not limited to intradermal, intramuscular, intraperitoneal, intravenous, subcutaneous, intranasal, epidural, locoregional administration as described in WO2015158924 and oral routes. In a particular embodiment, the administration is via the intravenous or intramuscular route. The vector of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the pharmaceutical composition of the invention locally to the area in need of treatment, e.g. the liver or the muscle. This may be achieved, for example, by means of an implant, said implant being of a porous, nonporous, or gelatinous material, including membranes, such as sialastic membranes, or fibers.

The amount of the vector of the invention which will be effective in the treatment of disorder to be treated can be determined by standard clinical techniques. In addition, in vivo and/or in vitro assays may optionally be employed to help predict optimal dosage ranges. The precise dose to be employed in the formulation will also depend on the route of administration, and the seriousness of the disease, and should be decided according to the judgment of the practitioner and each patient's circumstances. The dosage of the vector of the invention administered to the subject in need thereof will vary based on several factors including, without limitation, the route of administration, the specific disease treated, the subject's age or the level of expression necessary to obtain the therapeutic effect. One skilled in the art can readily determine, based on its knowledge in this field, the dosage range required based on these factors and others. In case of a treatment comprising administering an AAV vector to the subject, typical doses of the vector are of at least 1×10⁸ vector genomes per kilogram body weight (vg/kg), such as at least 1×10⁹ vg/kg, at least 1×10¹⁰ vg/kg, at least 1×10¹¹ vg/kg, at least 1×10¹² vg/kg at least 1×10¹³ vg/kg, at least 1×10¹⁴ vg/kg or at least 1×10¹⁵ vg/kg.

In a particular embodiment, the vector of the invention may be administered at a dose lower than typical doses used in gene therapy. In particular, in a treatment comprising administering an AAV vector to the subject in need thereof, the vector may be administered at a dose at least 2-times lower than the above typical doses, in particular at a dose at least 3-times, 4-times, 5-times, 6-times, 7-times, 8-times, 9-times, 10-times, 11-times, 12-times, 13-times, 14-times, 15-times, 16-times, 17-times, 18-times, 19-times, 20-times, 21-times, 22-times, 23-times, 24-times, 25-times, 26-times, 27-times, 28-times, 29-times, 30-times, 31-times, 32-times, 33-times, 34-times, 35-times, 36-times, 37-times, 38-times, 39-times, 40-times, 41-times, 42-times, 43-times, 44-times, 45-times, 46-times, 47-times, 48-times, 49-times, or even at least 50-times lower than the typical AAV doses typically used in gene therapy.

### EXAMPLES

Materials and Methods

In Vivo Studies

All mouse studies were performed according to the French and European legislation on animal care and experimentation (2010/63/EU) and approved by the local institutional ethical committee (protocol no. 2016-002B). AAV vectors were administered intravenously via the tail vein to 6-8 week-old male C57B16/J mice. PBS injected littermates were used as controls. At sacrifice mice were perfused with PBS to avoid blood contamination in tissues. After sampling tissues were homogenized in DNAse/RNAse free water using Fastprep tubes (4 m/s; 60 secondes).

mSeAP Activity

mSeAP activity in tissues was measured using the Phospha-Light™ SEAP Reporter Gene Assay System (ThermoFisher) following manufacturer's instructions.

Plasmids Construction.

Enhancer/promoter (EP) sequences were purchased from a commercial source. mSeAP cDNA, was ligated to each EP sequence using XhoI and Mlu1 restriction enzymes. Resulting transgene expression cassettes were cloned between two ITRs derived from AAV2 using the XbaI restriction sites flanking the sequence.

A description of the promoter/enhancer combinations used in the experimental part is made in Table 1 below.

TABLE 1
Description of the promoter/enhancer combinations used in the figures

| Name used in Figures | SEQ ID NO | Enhancer | Promoter |
|---|---|---|---|
| spC5-12 | 2 | — | spC5-12¹ |
| MCK-spC5-12 | 35 | MCK² | spC5-12¹ |
| H1-MCK-spC5-12 | 10 | H1³, MCK² | spC5-12¹ |
| H3-MCK-spC5-12 | 12 | H3⁴, MCK² | spC5-12¹ |
| H3-spC5-12 | 11 | H3⁴ | spC5-12¹ |
| CK6 | 6 | — | CK6⁵ |
| H3-CK6 | 36 | H3⁴ | CK6⁵ |
| CK8 | 7 | — | CK8⁶ |
| H3-CK8 | 37 | H3⁴ | CK8⁶ |
| H3-ACTA1 | 38 | H3⁴ | ACTA1⁷ |
| F-spC5-12 | 39 | F⁸ | spC5-12¹ |

¹spC5-12 promoter (SEQ ID NO: 2);
²MCK enhancer (SEQ ID NO: 5);
³one copy of SEQ ID NO: 1;
⁴three copies of SEQ ID NO: 1;
⁵CK6 promoter (SEQ ID NO: 6);
⁶CK8 promoter (SEQ ID NO: 7);
⁷ACTA1 promoter (SEQ ID NO: 8);
⁸fibrinogen alpha chain enhancer (SEQ ID NO: 30).

Results

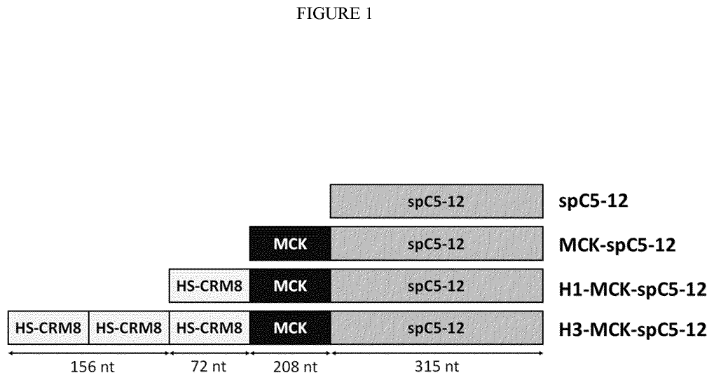

We evaluated the tissue specific expression driven by four combinations of enhancers and promoter as reported in FIG. 1. These promoters were composed of a muscle specific promoter (spC5-12) combined with the muscle enhancer MCK (MCK-spC5-12, Table 1). This combination of promoter and enhancer was known in the literature as E-Syn (Wang B. Gene therapy 2008). New hybrid promoters were obtained by the fusion of one (H1) or three repetitions (H3) of the hepatic enhancer HS-CRM8 at position 1 of the MCK-spC5-12 promoter/enhancer combination (H1-MCK-spC5-12 and H3-MCK-spC5-12 respectively, Table 1). To validate the tissue-specificity of these constructs we used the mouse secreted alkaline phosphatase (mSeAP) reporter gene. Transgene expression cassettes bearing this reporter gene and the four promoter/enhancer combinations were pseudotyped in AAV9 vectors produced by triple transfection and cesium chloride gradient purification.

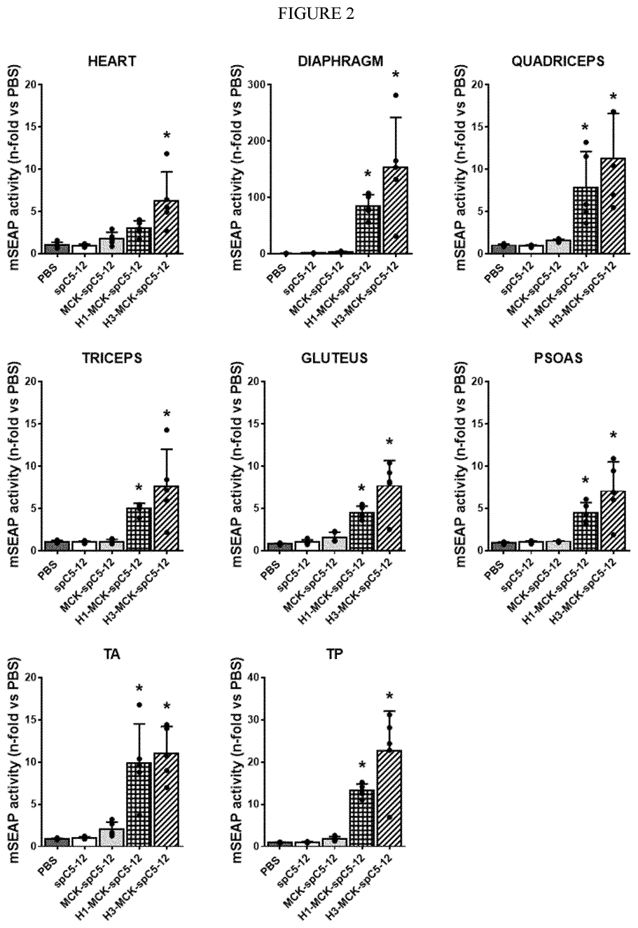

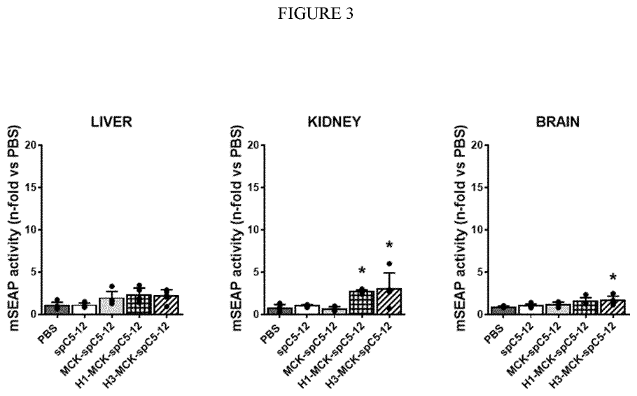

The mSeAP-AAV9 vectors were intravenously injected in two month-old C57B16/Jmale mice at the dose of 2×10¹¹ vector genome per mouse. In parallel mice were injected with phosphate buffer saline (PBS) as control. Animals were sacrificed 1 month after vectors injection. Mice were perfused with PBS to avoid blood contamination in tissues. Muscle and non-muscle tissues were biochemically analyzed to quantify mSeAP enzymatic activity. In muscle, the spC5-12 promoter or the MCK-spC5-12 enhancer/promoter did not lead to a significant and detectable mSEAP activity compared to PBS injected mice (FIG. 2). Interestingly, AAV9 expressing mSEAP under the transcriptional control of H1-MCK-spC5-12 and H3-MCK-spC5-12 hybrid promoters allowed for a significant, 5 to 10-fold increase in mSEAP activity in different skeletal muscles (FIG. 2). In diaphragm and tibialis posterior, we observed the higher increases in mSEAP activity, 150 and 20-fold respectively for the H3-MCK-spC5-12 enhancer/promoter. In liver we did not observe any significant increase in mSEAP expression (FIG. 3). In kidneys and brain, a significant 2 to 3-fold increase in mSEAP expression was observed in mice that received vectors carrying H1-MCK-spC5-12 and H3-MCK-spC5-12 (FIG. 3).

In view of the much higher mSEAP expression in muscles as compared to other tissues, these data demonstrate that the fusion of one or three copies of HS-CRM8 at the 5′ of a synthetic muscle promoter (MCK-spC5-12) specifically increases the expression of the transgene in muscle thus providing a new tool for gene therapy for neuromuscular disorders.

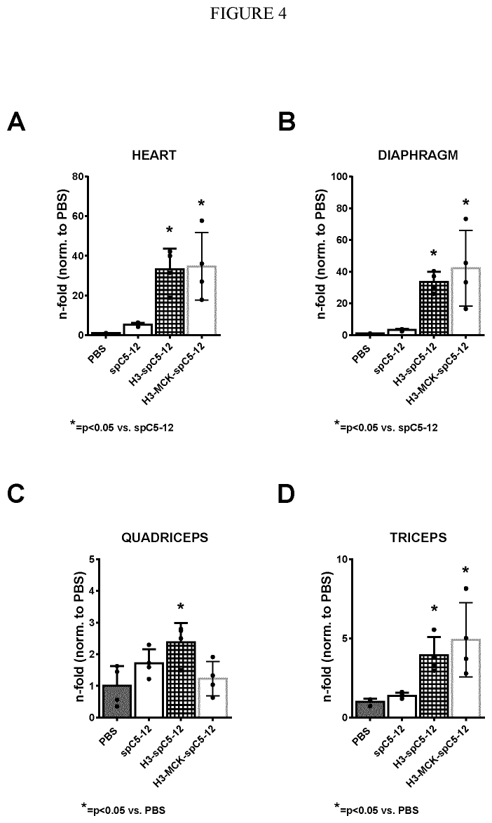

We then reduced the size of the promoter by removing the MCK enhancer. We prepared AAV9 vectors expressing mSEAP under the transcriptional control of (i) the spC5-12 promoter, (ii) the spC5-12 promoter fused directly with three copies of HS-CRM8 (H3-spC5-12), or (iii) the H3-MCK-spC5-12 hybrid promoter. The mSeAP-AAV9 vectors were injected in C57Bl/6 male mice at the dose of 4×10¹¹ vector genome per mouse. In parallel mice were injected with phosphate buffer saline (PBS) as control. Animals were sacrificed 1 month after vectors injection. Mice were perfused with PBS to avoid blood contamination in tissues. Muscle tissues were analyzed to quantify mSeAP enzymatic activity. Importantly, in muscles, differently from the spC5-12 promoter, the fusion of the spC5-12 promoter with H3 or H3-MCK led to significant and detectable mSEAP activity, when compared to PBS injected mice (FIG. 4). These data indicate that the increase in muscle expression of the transgene is not dependent on the presence of the MCK enhancer. The following transgene expression cassette constructs do not include this enhancer.

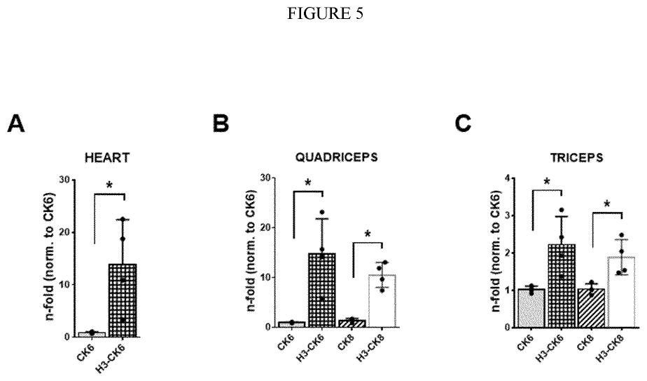

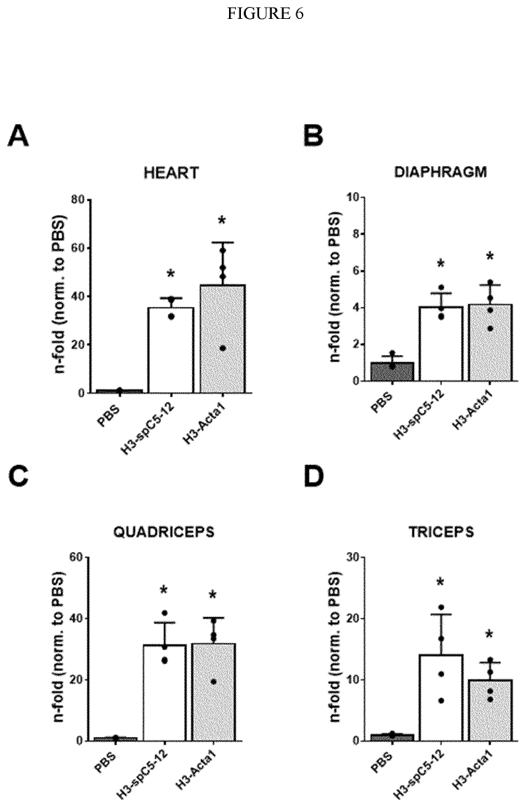

To confirm the robustness of the effect observed when the H3 enhancer is fused with muscle promoters, we created two new combinations of promoter/enhancer involving the CK6 and CK8 promoters, frequently used in in-vivo gene therapy. We prepared AAV9 vectors expressing mSEAP under the transcriptional control of the CK6 or CK8 promoter or under the control of CK6 or CK8 fused directly with three copies of HS-CRM8 (H3-CK6 and H3-CK8 respectively). The mSeAP-AAV9 vectors were injected in C57Bl/6 male mice at the dose of 5×10¹¹ vector genome per mouse. Animals were sacrificed fifteen days after vectors injection. Mice were perfused with PBS to avoid blood contamination in tissues. Muscle tissues were analyzed to quantify mSeAP enzymatic activity. Importantly, in muscles, the fusion of H3 with both CK6 and CK8 muscle specific promoters led to significant and detectable mSEAP activity compared to the parental CK6 and CK8 promoters respectively (FIG. 5). Similar findings were reported also for a different promoter, ACTA1 that, when fused with three copies of the HS-CRM8 (H3-ACTA1) led to levels of mSEAP transgene expression similar to those measured by H3-spC5-12 hybrid promoter in muscles (FIG. 6). Of note, in a different experimental setup, ACTA1 promoter showed an efficacy comparable to that of spC5-12 promoter in muscle (data not shown). These data indicate that H3 induces an increase in promoter efficacy regardless of the muscle-selective promoter.

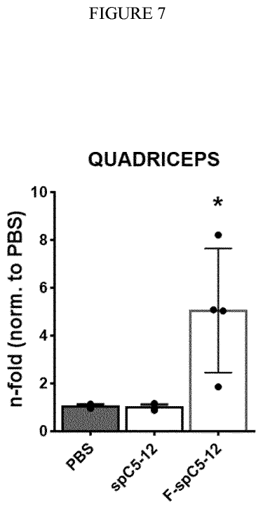

Finally, to confirm that other liver-selective enhancers have a similar effect, we tested the regulatory sequence controlling the transcription of fibrinogen alpha chain (described as HS-CRM11 in Chuah et al., Molecule Therapy, 2014, vol. 22, no. 9, p. 1605) fused with spC5-12 promoter (F-spC5-12). The F-spC5-12 hybrid promoter led to a significant increase in mSEAP transgene expression when compared to spC5-12 promoter (FIG. 7) thus indicating that, similarly to H3, other liver-specific enhancers increase the transgene expression driven by muscle-specific promoter in muscle.

1-19. (canceled)

20. A nucleic acid molecule comprising one or a plurality of liver-selective enhancer(s) operably linked to a muscle-selective promoter, wherein:
the liver-selective enhancer comprises a sequence selected from the group consisting of SEQ ID NO:1, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33, a functional variant having 80% identity to any one of the sequences selected from SEQ ID NO:1, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33, and a functional fragment thereof; or
the plurality of liver-selective enhancers comprises at least one liver-selective enhancer comprising a sequence selected from the group consisting of SEQ ID NO:1, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33, a functional variant having 80% identity to any one of the sequences selected from SEQ ID NO:1, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32 and SEQ ID NO:33, and a functional fragment thereof.

21. The nucleic acid molecule of claim 20, wherein all the liver-selective enhancers of the plurality of liver-selective enhancers have the same sequence.

22. The nucleic acid molecule of claim 20, wherein at least two of the liver-selective enhancers of the plurality of liver-selective enhancers have a different sequence.

23. The nucleic acid molecule of claim 20, wherein the plurality of liver-selective enhancers comprises at least two liver-selective enhancers.

24. The nucleic acid molecule of claim 20, wherein the plurality of liver-selective enhancers comprises three liver-selective enhancers.

25. The nucleic acid molecule of claim 20, wherein the sequence of the liver-selective enhancer consists of SEQ ID NO:1, or is a functional variant having a sequence at least 80% identical to SEQ ID NO:1.

26. The nucleic acid molecule of claim 20, wherein the sequence of the liver-selective enhancer consists of SEQ ID NO:30, or is a functional variant having a sequence at least 80% identical to SEQ ID NO:30.

27. The nucleic acid molecule of claim 20, wherein the promoter is a spC5-12 promoter.

28. The nucleic acid molecule of claim 27, wherein the spC5-12 promoter consists of the sequence shown in SEQ ID NO: 2, 3 or 4, or a functional variant having a sequence that is at least 80% identical to SEQ ID NO: 2, 3 or 4.

29. The nucleic acid molecule of claim 20, wherein the promoter is a CK6 promoter.

30. The nucleic acid molecule of claim 29, wherein the CK6 promoter consists of the sequence shown in SEQ ID NO:6, or a functional variant having a sequence that is at least 80% identical to SEQ ID NO:6.

31. The nucleic acid molecule of claim 20, wherein the promoter is a CK8 promoter.

32. The nucleic acid molecule of claim 31, wherein the CK8 promoter consists of the sequence shown in SEQ ID NO:7, or a functional variant having a sequence that is at least 80% identical to SEQ ID NO:7.

33. The nucleic acid molecule of claim 20, wherein the promoter is a ACTA1 promoter.

34. The nucleic acid molecule of claim 33, wherein the ACTA1 promoter consists of the sequence shown in SEQ ID NO:8, or a functional variant having a sequence that is at least 80% identical to SEQ ID NO:8.

35. The nucleic acid molecule of claim 20, further comprising a muscle-selective enhancer located between the liver-selective enhancer, or the plurality of liver-selective enhancers, and the muscle-selective promoter.

36. An expression cassette comprising the nucleic acid molecule of claim 20 operably linked to a transgene of interest.

37. A vector comprising the expression cassette according to claim 36, wherein said vector is a plasmid or viral vector.

38. The vector of claim 37, wherein said viral vector is an adeno-associated virus (AAV) vector.

39. An isolated recombinant cell comprising the expression cassette according to claim 36.

40. A method of treating a neuromuscular disorder comprising the administration of an expression cassette according to claim 36, a vector comprising said expression cassette, or a recombinant cell comprising said expression cassette to a subject in need of treatment.

41. The method of claim 40, wherein the neuromuscular disorder is selected from the group consisting of muscular dystrophies, myotonic dystrophy (Steinert disease), Duchenne muscular dystrophy, Becker muscular dystrophy, limb-girdle muscular dystrophy, facioscapulohumeral muscular dystrophy, congenital muscular dystrophy, oculopharyngeal muscular dystrophy, distal muscular dystrophy, Emery-Dreifuss muscular dystrophy, motor neuron diseases, amyotrophic lateral sclerosis (ALS), spinal muscular atrophy, Infantile progressive spinal muscular atrophy (type 1, Werdnig-Hoffmann disease), intermediate spinal muscular atrophy (Type 2), juvenile spinal muscular atrophy (Type 3, Kugelberg-Welander disease), adult spinal muscular atrophy (Type 4), spinal-bulbar muscular atrophy (Kennedy disease), inflammatory myopathies, polymyositis dermatomyositis, inclusion-body myositis, diseases of neuromuscular junction, myasthenia gravis, Lambert-Eaton (myasthenic) syndrome, congenital myasthenic syndromes, diseases of peripheral nerves, Charcot-Marie-Tooth disease, Friedreich's ataxia, Dejerine-Sottas disease, metabolic diseases of muscle, phosphorylase deficiency (McArdle disease), acid maltase deficiency (Pompe disease), phosphofructokinase deficiency (Tarui disease), debrancher enzyme deficiency (Cori or Forbes disease), mitochondrial myopathy, carnitine deficiency, carnitine palmityl transferase deficiency, phosphogly cerate kinase deficiency, phosphoglycerate mutase deficiency, lactate dehydrogenase deficiency, myoadenylate deaminase deficiency, myopathies due to endocrine abnormalities, hyperthyroid myopathy, hypothyroid myopathy, myotonia congenita, paramyotonia congenita, central core disease, nemaline myopathy, myotubular myopathy, and periodic paralysis.


SEQUENCE LISTING (39 sequences)

SEQ ID NO: 1 - DNA, 72 bp, artificial
  HS-CRM8 (x1)
gggggaggctgctggtgaatattaaccaaggtcaccccagttatcggaggagcaaacaggggctaagtccac

SEQ ID NO: 2 - DNA, 315 bp, artificial
  spC5.12 (1)
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 3 - DNA, 400 bp, artificial
  spC5.12 (2)
ccgagctccaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatcccccgggctgcaggaattcgatat

SEQ ID NO: 4 - DNA, 358 bp, artificial
  spC5.12 (3)
caccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgacggttcctcacccgtcgccatatttgggtgtccgccctcggccggggccgcattcctgggggccgggcggtgctcccgcccgcctcgataaaaggctccggggccggcggcggcccacgagctacccggaggagcgggaggcgccaagctctagaactagtggatct

SEQ ID NO: 5 - DNA, 208 bp, artificial
  MCK enhancer
cactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgg

SEQ ID NO: 6 - DNA, 575 bp, artificial
  CK6 promoter
ctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 7 - DNA, 454 bp, artificial
  CK8 promoter
ctacaaacgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgccctcattctaccaccacctccacagcacagacagacactcaggagccagccagc

SEQ ID NO: 8 - DNA, 324 bp, artificial
  Acta1 promoter
aaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 9 - DNA, 395 bp, artificial
  HS-CRM8 x 1 - spC5.12
gggggaggctgctggtgaatattaaccaaggtcaccccagttatcggaggagcaaacaggggctaagtccacaagcttcacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 10 - DNA, 601 bp, artificial
  HS-CRM8 x 1 - MCK enhancer - spC5.12
gggggaggctgctggtgaatattaaccaaggtcaccccagttatcggaggagcaaacaggggctaagtccacaagcttcactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 11 - DNA, 545 bp, artificial
  HS-CRM8x3 - spC5.12
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacaagcttcacaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 12 - DNA, 751 bp, artificial
  HS-CRM8x3 - MCK enhancer - spC5.12
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacaagcttcactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 13 - DNA, 91 bp, artificial
  SV40 intron
ctctaaggtaaatataaaatttttaagtgtataatgtgttaaactactgattctaattgtttgtgtattttagattccaacctatggaact

SEQ ID NO: 14 - DNA, 366 bp, artificial
  MCK promoter
caatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 15 - DNA, 1060 bp, artificial
  desmin promoter
taccccctgccccccacagctcctctcctgtgccttgtttcccagccatgcgttctcctctataaatacccgctctggtatttggggttggcagctgttgctgccagggagatggttgggttgacatgcggctcctgacaaaacacaaacccctggtgtgtgtgggcgtgggtggtgtgagtagggggatgaatcagggagggggcgggggacccagggggcaggagccacacaaagtctgtgcgggggtgggagcgcacatagcaattggaaactgaaagcttatcagaccctttctggaaatcagcccactgtttataaacttgaggccccaccctcgacagtaccggggaggaagagggcctgcactagtccagagggaaactgaggctcagggccagctcgcccatagacatacatggcaggcaggctttggccaggatccctccgcctgccaggcgtctccctgccctcccttcctgcctagagacccccaccctcaagcctggctggtctttgcctgagacccaaacctcttcgacttcaagagaatatttaggaacaaggtggtttagggcctttcctgggaacaggccttgaccctttaagaaatgacccaaagtctctccttgaccaaaaaggggaccctcaaactaaagggaagcctctcttctgctgtctcccctgaccccactcccccccaccccaggacgaggagataaccagggctgaaagaggcccgcctgggggctgcagacatgcttgctgcctgccctggcgaaggattggtaggcttgcccgtcacaggacccccgctggctgactcaggggcgcaggcctcttgcgggggagctggcctccccgcccccacggccacgggccgccctttcctggcaggacagcgggatcttgcagctgtcaggggaggggaggcgggggctgatgtcaggagggatacaaatagtgccgacggctgggggccctgtctcccctcgccgcatccactctccggccggccgcctgcccgccgcctcctccgtgcgcccgccagcctcgcccg

SEQ ID NO: 16 - DNA, 195 bp, artificial
  Unc45b promoter
tttctcattctagaaagtaccagtcaactctccaccagcccagctgttggcagacgcacacctccatccccctgccctcagacatttgccactgatttctcagctgtcatcccctctccataaatagaccctatcagagaaagtccattgcactaatataaggggtgaccacatttctacaaaaccacaattaat

SEQ ID NO: 17 - DNA, 101 bp, artificial
  linker
cgatgggcaactcatgcaattattgtgagcaatacacacgcgcttccagcggagtataaatgcctaaagtaataaaaccgagcaatccatttacgaatgtt

SEQ ID NO: 18 - DNA, 300 bp, artificial
  linker
cgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagta

SEQ ID NO: 19 - DNA, 500 bp, artificial
  linker
cgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagt

SEQ ID NO: 20 - DNA, 1000 bp, artificial
  linker
cgatgggcaactcatgcaattattgtgagcaatacacacgcgcttccagcggagtataaatgcctaaagtaataaaaccgagcaatccatttacgaatgtttgctgggtttctgttttaacaacattttctgcgccgccacaaattttggctgcatcgacagttttcttctgcccaattccagaaacgaagaaatgatgggtgatggtttcctttggtgctactgctgccggtttgttttgaacagtaaacgtctgttgagcacatcctgtaataagcagggccagcgcagtagcgagtagcatttttttcatggtgttattcccgatgctttttgaagttcgcagaatcgtatgtgtagaaaattaaacaaaccctaaacaatgagttgaaatttcatattgttaatatttattaatgtatgtcaggtgcgatgaatcgtcattgtattcccggattaactatgtccacagccctgacggggaacttctctgcgggagtgtccgggaataattaaaacgatgcacacagggtttagcgcgtacacgtattgcattatgccaacgccccggtgctgacacggaagaaaccggacgttatgatttagcgtggaaagatttgtgtagtgttctgaatgctctcagtaaatagtaatgaattatcaaaggtatagtaatatcttttatgttcatggatatttgtaacccatcggaaaactcctgctttagcaagattttccctgtattgctgaaatgtgatttctcttgatttcaacctatcataggacgtttctataagatgcgtgtttcttgagaatttaacatttacaacctttttaagtccttttattaacacggtgttatcgttttctaacacgatgtgaatattatctgtggctagatagtaaatataatgtgagacgttgtgacgttttagttcagaataaaacaattcacagtctaaatcttttcgcacttgatcgaatatttctttaaaaatgg

SEQ ID NO: 21 - DNA, 101 bp, artificial
  HS-CRM1
cagccaatgaaatacaaagatgagtctagttaataatctacaattattggttaaagaagtatattagtgctaatttccctccgtttgtcctagcttttctc

SEQ ID NO: 22 - DNA, 71 bp, artificial
  HS-CRM2
tgaatgaccttcagcctgttcccgtccctgatatgggcaaacattgcaagcagcaaacagcaaacacatag

SEQ ID NO: 23 - DNA, 173 bp, artificial
  HS-CRM3
ggcgtattcttaagaatagattaaataatcataaaaagatctatacttaaaaattgaaaaatgcttaaatattaaaattcttctcataaaaaaatactaatttaaaaatgagcctgaaatgtttatctatttattgcacagggttgcatacataaaacgacacaccctcttgt

SEQ ID NO: 24 - DNA, 551 bp, artificial
  HS-CRM4
agtttggaacaagactatataccatatcctacaggaagaataaaagtaaaggaaaggtgccatctctactgaatagagagtcctaacaaaaaggcttcaaaaggactctgcatctttaataatataaaaaggctaggacacaaacagcatcatctaaaatgccattagaaatacttcacatacaaaaaggtctaagtaaagcaggattttataaagtgatcaaaaaagaaacactaagggggaaaaatcttttaagattaaagaggtttttcaaaggacaagttgaagtggctgtaaaatttatgaggcagcattaaacttcagttctaagtaacaataaattattcaccataaaaacatacatgtgtcaaatattataagcctcttaaactttttaaaacaatttcttgcagaactgattagatatattaagtcaagattagcagatactaactttttcattagcatactatgatcactcagagtaaaggaggaaatttagaaaagaaataagacagaaccatcaatagtcgattcaccaccaaatgtga

SEQ ID NO: 25 - DNA, 141 bp, artificial
  HS-CRM5
tgcgggaatcagcctttgaaacgatggccaacagcagctaataataaaccagtaatttgggatagacgagtagcaagagggcattggttggtgggtcaccctccttctcagaacacattataaaaaccttccgtttccaca

SEQ ID NO: 26 - DNA, 135 bp, artificial
  HS-CRM6
gcatgattttaaggactggttgtttatgagccaatcagaggtgttgaataaacacctccctactaggtcaaggtagaaaggggagggcaaatattggaaaaaaaaaacatgatgagaagtctataaaaattgtgt

SEQ ID NO: 27 - DNA, 94 bp, artificial
  HS-CRM7
ctaaaatgggcaaacattgcaagcagcaaacagcaaacacacagccctccctgcctgctgaccttggagctggggcagaggtcagagacctctc

SEQ ID NO: 28 - DNA, 171 bp, artificial
  HS-CRM9
aggaggaactgctcaaaacagacagaggctctttgtttgctttgcttctgtgtcaactgggcaacatttggaaacaacaaatattggttcagaggcccactgctttcttacccacctcctgctggtcagcttttccagctttcctgcacgtacacacaagcgcagctattt

SEQ ID NO: 29 - DNA, 170 bp, artificial
  HS-CRM10
cgatgctctaatctctctagacaaggttcatatttgtatgggttacttattctctctttgttgactaagtcaataatcagaatcagcaggtttgcagtcagattggcagggataagcagcctagctcaggagaagtgagtataaaagccccaggctgggagcagccatca

SEQ ID NO: 30 - DNA, 74 bp, artificial
  HS-CRM11
tgccactcctagttcccatcctatttaaatctgcaagaggtttggttaatcattggctttgtcctgtgtagaca

SEQ ID NO: 31 - DNA, 441 bp, artificial
  HS-CRM12
ttccttcccccttccaagacccccctgaatcctatcaaaagcacatcttccattcattgcttcccggtgtcattatgacaagcggctacaaatcaatagcagagggaaaggcaggaccaacccgcactcaccaagtgataaagattcactctcagccccgatttgctaatagcccataatagcagccattggcgccccgcattaaataatacatttcactccgcgtttattatgggatttttaaaactcctcaccaaattggattttctcgatggtctctaatttccacatttatcatttaaaattaaactgctctgtggaaaggggggatagagaagaagaaggtagagagaggccagacagtactgtatttttccttttgactcccccctttatgaaaacccataaataatatcaggtatcacagctataagcagcagg

SEQ ID NO: 32 - DNA, 88 bp, artificial
  HS-CRM13
ggagttgctggtgcttccccaggctggagattgagttaatattaacaggcccaaggcgatgtgggcttgtgcaatcataggcccggcc

SEQ ID NO: 33 - DNA, 41 bp, artificial
  HS-CRM14
atcgccaggtcacctgaggagttaatgaatacatatctcct

SEQ ID NO: 34 - DNA, 200 bp, artificial
  SA195 enhancer
actagttgagtaagtgaaaaaataataatctgtaaaaatgatgaatcccaatgactcacatgttgcaaaataactaggaagcaaaagggaaattagactttaaagagcgtaatcagatgcaagaaatgttggcttgtaggtggttaactaaaatcgcttacgggaagctcagacagctggggaatcctgatttagtagac

SEQ ID NO: 35 - DNA, 523 bp, artificial
  MCK enhancer - spC5.12
cactacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtggcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc

SEQ ID NO: 36 - DNA, 803 bp, artificial
  HS-CRM8x3 - CK6
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtctacgggtctaggctgcccatgtaaggaggcaaggcctggggacacccgagatgcctggttataattaaccccaacacctgctgcccccccccccccaacacctgctgcctgagcctgagcggttaccccaccccggtgcctgggtcttaggctctgtacaccatggaggagaagctcgctctaaaaataaccctgtccctggtgggcccaatcaaggctgtgggggactgagggcaggctgtaacaggcttgggggccagggcttatacgtgcctgggactcccaaagtattactgttccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctggggacagcccctcctggctagtcacaccctgtaggctcctctatataacccaggggcacaggggctgcccccgggtcac

SEQ ID NO: 37 - DNA, 563 bp, artificial
  HS-CRM8x3 - CK8
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtctacaaacgctagcatgctgcccatgtaaggaggcaaggcctggggacacccgagatgcctggttataattaacccagacatgtggctgcccccccccccccaacacctgctgcctctaaaaataaccctgcatgccatgttcccggcgaagggccagctgtcccccgccagctagactcagcacttagtttaggaaccagtgagcaagtcagcccttggggcagcccatacaaggccatggggctgggcaagctgcacgcctgggtccggggtgggcacggtgcccgggcaacgagctgaaagctcatctgctctcaggggcccctccctgggg

SEQ ID NO: 38 - DNA, 552 bp, artificial
  HS-CRM8x3 - Acta1
gggggaggctgctggtgaatattaaccaaggtcaccccagttatcggaggagcaaacaggggctaagtccacaagcttgggggaggctgctggtgaatattaaccaaggtcaccccagttatcggaggagcaaacaggggctaagtccacgggggaggctgctggtgaatattaaccaaggtcaccccagttatcggaggagcaaacaggggctaagtccacactagtaaaggcatagccccatatatcagtgatataaatagaacctgcagcaggctctggtaaatgatgactacaaggtggactgggaggcagcccggccttggcaggcatcgaccgggccaacccgctccttctttggtcaacgcaggggacccgggcgggggcccaggccgcgaaccggccgagggagggggctctagtgcccaacacccaaatatggctcgagaagggcagcgacattcctgcggggtggcgcggagggaatgcccgcgggctatataaaacctgagcagagggacaagcggccaccgcagcggacagcgccaagtg

SEQ ID NO: 39 - DNA, 400 bp, artificial
  HS-CRM11 - spC5-12
tgccactcctagttcccatcctatttaaatctgcaagaggtttggttaatcattggctttgtcctgtgtagacaactagtctagtcaccgcggtggcggccgtccgccctcggcaccatcctcacgacacccaaatatggcgacgggtgaggaatggtggggagttatttttagagcggtgaggaaggtgggcaggcagcaggtgttggcgctctaaaaataactcccgggagttatttttagagcggaggaatggtggacacccaaatatggcgaccggttcctcaaccggtcgccatatttgggtgtccgccctcggccggggccgcattcctgggggccgggcggtgctcccgcccgcctcgataaaaggctccggggccggcggcggcccacgagc